(19) United States
(12) Patent Application Publication
(10) Pub. No.: US 2004/0067918 A1
(43) Pub. Date: Apr. 8, 2004

(54) Combination of an aldosterone receptor antagonist and nicotinic acid or a nicotinic acid derivative

(76) Inventors: Bradley T. Keller, Chesterfield, MO (US); Ellen G. McMahon, Sunset Hills, MO (US); Elaine S. Krul, Warson Woods, MO (US)

Correspondence Address: Pharmacia Corporation, Global Patent Department, P.O. Box 1027, Chesterfield, MO, 63006, US

(21) Appl. No.: 10/391,212

(22) Filed: Mar. 18, 2003

Related U.S. Application Data
(60) Provisional application No. 60/365,269, filed on Mar. 18, 2002.

(51) Int. Cl. A61K 31/57; A61K 31/455

(52) U.S. Cl. 514/171; 514/355; 514/356

(57) ABSTRACT

Novel methods and combinations for the treatment and/or prophylaxis of a pathologic condition in a subject, wherein the methods comprise the administration of one or more aldosterone receptor antagonists and one or more, nicotinic acid derivatives and the combinations comprise one or more of said aldosterone receptor antagonists and one or more of said nicotinic acid derivatives.

### BACKGROUND OF THE INVENTION

1. Field of the Invention

The present invention relates to methods for the treatment and/or prophylaxis of one or more pathogenic conditions in a subject arising from or exacerbated by endogenous mineralocorticoid activity, especially in the presence of dyslipidemia or in a subject susceptible to or suffering from dyslipidemia. Particularly, the invention relates to the use of an aldosterone receptor antagonist combined with the use of nicotinic acid or a nicotinic acid derivative for the treatment or prevention of one or more pathogenic conditions selected from, but not limited to, cardiovascular-related conditions, inflammation-related conditions, neurological-related conditions, musculo-skeletal-related conditions, metabolism-related conditions, endocrine-related conditions, dermatologic-related conditions and cancer-related conditions. More particularly, the invention relates to treating or preventing one or more of said conditions with said combination therapy, wherein the aldosterone receptor antagonist is an epoxy-steroidal compound, such as eplerenone.

2. Description of the Related Art

Aldosterone Receptor Antagonists

Aldosterone is the body's most potent known mineralocorticoid hormone. As connoted by the term mineralocorticoid, this steroid hormone has mineral-regulating activity. It promotes sodium (Na⁺) reabsorption in epithelial cells through binding and activating the mineralocorticoid receptor (MR). Aldosterone increases sodium and water reabsorption in the distal nephron and promotes potassium (K⁺) and magnesium (Mg²⁺) excretion.

Aldosterone also can produce responses in nonepithelial cells. In fact, aldosterone receptors have been recently identified in brain tissue, heart tissue and blood vessels. These aldosterone-mediated responses can have adverse consequences on the structure and function of the cardiovascular system. Hence, inappropriate aldosterone exposure can contribute to organ damage in disease settings.

The effect of aldosterone can be reduced through the use of an aldosterone receptor antagonist. A number of aldosterone receptor blocking compounds have been disclosed in the literature. For example, one commercially available aldosterone receptor antagonist is spironolactone (also known as ALDACTONE® (Pharmacia, Chicago, Ill.)). According to United States Pharmacopeia, Rockville, Md., spironolactone is indicated for the management of essential hypertension, primary aldosteronism, hypokalemia, and edematous conditions such as congestive heart failure, cirrhosis of the liver, and nephrotic syndrome. The administration of spironolactone to severe heart failure patients was evaluated in the Randomized Aldactone Evaluation Study (RALES). RALES was a randomized, double-blinded, placebo-controlled trial that enrolled participants who had severe heart failure and a left ventricular ejection fraction of no more than 35% and who were receiving standard therapy, which typically included an angiotensin-converting enzyme inhibitor, a loop diuretic, and, in some cases, digoxin. The RALES subjects treated with spironolactone had a statistically significant reduction in mortality and incidence of hospitalization relative to placebo-treated subjects. New England Journal of Medicine 341, 709-717 (1999).

Another class of steroidal-type aldosterone receptor antagonists exemplified by epoxy-containing spirolactone derivatives is described in U.S. Pat. No. 4,559,332 issued to Grob et al. This patent describes 9α, 11α-epoxy-containing spirolactone derivatives as aldosterone receptor antagonists that are useful for the treatment of hypertension, cardiac insufficiency and cirrhosis of the liver. One of the epoxy-steroidal aldosterone antagonist compounds described in U.S. Pat. 4,559,332 is eplerenone (also known as epoxymexrenone). Eplerenone is an aldosterone receptor antagonist that has a higher specificity for the MR compared to spironolactone.

Another class of steroidal-type aldosterone receptor antagonists is exemplified by drospirenone. Developed by Schering AG, this compound is an antagonist of mineralocorticoid and androgenic receptors, while also possessing progestagenic characteristics.

Additional uses of aldosterone receptor antagonists have been disclosed in the literature. For example, WO 01/95892 and WO01/95893 are directed to methods for the treatment or prophylaxis of aldosterone-mediated pathogenic effects in a subject using an aldosterone receptor antagonist. WO02/09683 is directed to methods of using aldosterone antagonists to mediate inflammation.

Therapies comprising the administration of an aldosterone receptor antagonist in combination with several other pharmacologically active compounds have been reported in the literature.

MacLaughlan, et al., WO96/40258, disclose a combination therapy treatment utilizing spironolactone and angiotensin II receptor antagonist for treating congestive heart failure.

Egan et al., WO 96/40255, disclose a combination treatment therapy utilizing an epoxy-steroidal aldosterone receptor antagonist and an angiotensin II antagonist for treating cardiofibrosis.

Alexander et al., WO 96/40257, disclose a combination treatment therapy utilizing an epoxy-steroidal aldosterone receptor antagonist and an angiotensin II antagonist for treating congestive heart failure.

Perez et al., WO 00/27380, disclose a combination treatment therapy utilizing an angiotensin converting enzyme inhibitor and an aldosterone receptor antagonist for reducing morbidity and mortality resulting from cardiovascular disease.

Alexander et al., WO 00/51642, disclose a combination treatment therapy utilizing an angiotensin converting enzyme inhibitor and an epoxy-steroidal aldosterone receptor antagonist for treating cardiovascular disease.

Alexander et al., WO 02/09760, disclose a combination therapy utilizing an epoxy-steroidal aldosterone receptor antagonist and a beta-adrenergic antagonist for treating circulatory disorders, including cardiovascular disorders such as hypertension, congestive heart failure, cirrhosis and ascites.

Schuh, WO 02/09761, disclose a combination treatment therapy utilizing an epoxy-steroidal aldosterone receptor antagonist and a calcium channel blocker for treating hypertension, congestive heart failure, cirrhosis and ascites.

Rocha et al., WO 02/09759, disclose a combination treatment therapy utilizing an epoxy-steroidal aldosterone receptor antagonist and a cyclooxygenase-2 inhibitor for treating inflammation related cardiovascular disorders.

Keller, et al., WO 03/07993, disclose a combination treatment therapy utilizing an aldosterone receptor antagonist and an HMG-CoA reductase inhibitor for treating or preventing pathological conditions.

U.S. Pat. 5,569,652 discloses a combination of the aldosterone receptor antagonist drospirenone and an estrogen for use as an oral contraceptive.

Nicotinic Acid Derivatives

Nicotinic acid and derivatives of nicotinic acid comprise a class of drugs that have effects on lipoprotein levels. Nicotinic acid (niacin) is a B-complex vitamin reported as early as 1955 to act as a hypolipidemic agent (R. Altschl, et al., Arch. Biochem. Biophys., 54, 558-9 (1955)). It is sometimes used to raise low HDL levels and lower VLDL and LDL levels. Useful commercial formulations of nicotinic acid include Niacor, Niaspan, Nicobid, Nicolar, Slo-Niacin. Nicotinic acid is contraindicated for patients having hepatic dysfunction, active peptic ulcer, or arterial bleeding. Another compound in this class useful for cardiovascular indications is niceritrol (T. Kazumi et al., Curr. Ther. Res., 55, 546-51). J. Sasaki et al. (Int. J. Clin. Pharm. Ther., 33 (7), 420-26 (1995)) describes a reduction in cholesterol ester transfer activity by niceritrol monotherapy. Acipimox (5-methyl pyrazine-2-carboxylic acid 4-oxide, U.S. Pat. No. 4,002,750) is structurally similar to nicotinic acid and has antihyperlipidemic activity. Another related drug, acifran (4,5-dihydro-5-methyl-4-oxo-5-phenyl-2-furancarboxylic acid, EP0006305), is structurally similar to nicotinic acid and has antihyperlipidemic activity. Cyclophenylhexyl derivatives of nicotinic acid are also useful in the treatment of dyslipidemias. For example, the 2-tert-butyl-4-cyclophenylhexyl ester of nicotinic acid (L44) and the 2-tert-butyl-4-cyclophenylhexyl ester 1-oxide of nicotinic acid (L44-O), are disclosed as hypolipidemic agents in U.S Pat. No. 4,321,268 and Drugs of the Future 12, 349-351 (1987).

Several combination therapies involving niacin have been described in the literature for the treatment of cardiovascular disease. Zema, (J. Am. Coll. Cardiol. 35, 640-646(2000)) describes alterations in lipid profiles of patients with hypoalphalipoproteinemia who are treated with a combination of gemfibrozil and niacin.

Keller, et al. (WO 00/38725) discloses a therapeutic combination comprising nicotinic acid and either an ileal bile acid transport inhibitor or a cholesteryl ester transport protein inhibitor.

Buntin, et al. (U.S. Pat. No. 4,759,923) disclose a method for lowering serum cholesterol by administering combination of a bile acid sequestering resin and niacin.

A combination therapy of fluvastatin and niceritrol is described by J. Sasaki et al. (Id.). Those researchers conclude that the combination of fluvastatin with niceritrol “at a dose of 750 mg/day dose does not appear to augment or attenuate beneficial effects of fluvastatin.”

L. Cashin-Hemphill et al. (J. Am. Med. Assoc., 264 (23), 3013-17 (1990)) describe the effects of a combination therapy of colestipol and niacin on coronary atherosclerosis. The described effects include nonprogression and regression in native coronary artery lesions.

A combination therapy of acipimox and simvastatin shows changes in HDL levels in patients having high triglyceride levels (N. Hoogerbrugge et al., J. Internal Med., 241, 151-55 (1997)).

Keller, et al. (WO 00/38729) claim a therapeutic combination comprising an ileal bile acid transport inhibitor and a nicotinic acid derivative.

Myers, et al. (U.S. Pat. No. 6,090,830) disclose a combination of an HMG CoA reductase inhibitor and niacin to lower lipids.

Dennick (U.S. Pat. No. 5,260, 305) discloses pharmaceutical combinations for treating dyslipidemias comprising pravastatin and either niacin or a related acid, such as acipimox, acifran, or a cyclohexylphenyl ester of nicotinic acid.

Dufresne (U.S. Pat. No. 5,260,332) discloses a combination of a squalene synthetase inhibitor and niacin as being useful in treating hypercholesterolemia.

Helms, et al. (U.S. Pat. No. 5,182,298) disclose a therapeutic combination comprising an inducer of the LDL receptor gene and niacin.

Ginsberg, “Update on the Treatment of Hypercholesterolemia, with a Focus on HMG Co-A Reductase Inhibitors and Combination Regimens”, Clin. Cardiol., Vol. 18(6), pp. 307-315 (June 1995), reports that, for resistant cases of hypercholesterolemia, therapy combining an HMG Co-A reductase inhibitor with either a bile acid sequestering resin, niacin or a nicotinic acid derivative generally is effective and well tolerated.

ADVICOR®, a newly approved prescription medicine (Dec. 17 2001, NDA 21-249) comprising a combination of niacin (500, 750, or 1000 mg/tablet) and lovastatin (20 mg/tablet), is marketed by Kos Pharmaceuticals.

Improved drug therapies for the treatment of subjects suffering from or susceptible to a pathological condition are highly desirable. In particular, there still is a need for drug therapies that (1) provide better control over pathological conditions, (2) further reduce pathological risk factors, (3) provide improved treatment and/or prevention of pathological conditions, (4) are effective in a greater proportion of subjects suffering from or susceptible to a pathological condition, particularly in those subjects who do not satisfactorily respond to conventional drug therapies, and/or (5) provide an improved side-effect profile relative to conventional drug therapies.

### SUMMARY OF THE INVENTION

In a first aspect, the invention is directed to methods for the treatment and/or prophylaxis of one or more pathogenic conditions in a subject arising from or exacerbated by endogenous mineralocorticoid activity, wherein the method comprises administering a therapeutically effective amount of an aldosterone receptor antagonist and nicotinic acid or a nicotinic acid derivative.

In another aspect, the invention is directed to methods for the treatment of one or more pathogenic conditions selected from the group consisting of cardiovascular-related conditions, inflammation-related conditions, neurological-related conditions, musculo-skeletal-related conditions, metabolism-related conditions, endocrine-related conditions, dermatologic-related conditions and cancer-related conditions, methods comprising administering a therapeutically effective amount of an aldosterone receptor antagonist and nicotinic acid or a nicotinic acid derivative.

In still another aspect, the invention is directed to methods of treating one or more of said conditions with said combination therapy, wherein the aldosterone receptor antagonist is an epoxy-steroidal compound such as eplerenone.

In still another aspect, the invention is directed to methods of treating one or more of said conditions with said combination therapy, wherein the aldosterone receptor antagonist is a spirolactone compound such as spironolactone.

In still another aspect, the invention is directed to combinations, including pharmaceutical compositions, comprising one or more aldosterone receptor antagonists and one or more compounds selected from the group consisting of nicotinic acid and nicotinic acid derivatives.

In still another aspect, the invention is directed to combinations comprising one or more compounds selected from the group consisting of nicotinic acid and nicotinic acid derivatives, and one or more aldosterone receptor antagonists, wherein at least one of said antagonists is an epoxy-steroidal compound such as eplerenone.

In still another aspect, the invention is directed to combinations comprising one or more compounds selected from the group consisting of nicotinic acid and nicotinic acid derivatives, and one or more aldosterone receptor antagonists, wherein at least one of said antagonists is a spirolactone compound such as spironolactone.

In still another aspect, the invention is directed to kits comprising one or more aldosterone receptor antagonists and one or more compounds selected from the group consisting of nicotinic acid and nicotinic acid derivatives.

In still another aspect, the invention is directed to the preparation of a medicament comprising one or more aldosterone receptor antagonists and one or more compounds selected from the group consisting of nicotinic acid and nicotinic acid derivatives.

Other aspects of the invention will be in part apparent and in part pointed out hereinafter.

### DETAILED DESCRIPTION OF THE PREFERRED EMBODIMENTS

Improved drug therapies, especially for patients who do not satisfactorily respond to conventional drug therapies, are highly desirable. Further, the increasing prevalence of pathogenic conditions, particularly conditions selected from the group consisting of cardiovascular-related conditions, inflammation-related conditions, neurological-related conditions, musculo-skeletal-related conditions, metabolism-related conditions, endocrine-related conditions, dermatologic-related conditions and cancer-related conditions, suggests that newer therapeutic interventions and strategies are needed to replace or complement current approaches. The present invention addresses this need and provides a new drug therapy comprising the administration of one or more compounds that are aldosterone antagonists combined with the use of one or more compounds that are selected from the group consisting of nicotinic acid and nicotinic acid derivatives, for the treatment of one or more of said pathogenic conditions arising from or exacerbated by endogenous mineralocorticoid activity in a population of subjects characterized by or susceptible to dyslipidemia.

It has been discovered that the administration to a subject of one or more aldosterone receptor antagonists (e.g., those aldosterone receptor antagonists selected from the specific group consisting of compounds described below) and one or more compounds selected from the group consisting of nicotinic acid and nicotinic acid derivatives (e.g., those nicotinic acid derivatives selected from the specific group consisting of compounds described below) provides improved results in the prophylaxis and/or treatment of one or more pathogenic conditions in a subject arising from or exacerbated by endogenous mineralocorticoid activity, especially in the presence of dyslipidemia or in a subject susceptible to or suffering from dyslipidemia. Particularly, the invention relates to the use of an aldosterone receptor antagonist in combination with nicotinic acid or a nicotinic acid derivative for the treatment of one or more pathogenic conditions selected from the group consisting of cardiovascular-related conditions, inflammation-related conditions, neurological-related conditions, musculo-skeletal-related conditions, metabolism-related conditions, endocrine-related conditions, dermatologic-related conditions and cancer-related conditions.

The pathogenic conditions that can be treated or prevented in accordance with the present invention include, but are not limited to atherosclerosis, hypertension, cardiovascular disease, renal dysfunction, liver disease, cerebrovascular disease, vascular disease, retinopathy, neuropathy (such as peripheral neuropathy), insulinopathy, edema, endothelial dysfunction, baroreceptor dysfunction, migraine headaches, hot flashes, premenstrual tension, and the like.

Cardiovascular disease includes, but is not limited to, heart failure (such as congestive heart failure), arrhythmia, diastolic dysfunction (such as left ventricular diastolic dysfunction, diastolic heart failure, and impaired diastolic filling), systolic dysfunction, ischemia, hypertrophic cardiomyopathy, sudden cardiac death, myocardial and vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, fibrinoid necrosis of coronary arteries, and the like.

Renal dysfunction includes, but is not limited to, glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions of affecting glomeruli and microvessels), and the like.

Liver disease includes, but is not limited to, liver cirrhosis, liver ascites, hepatic congestion, and the like.

Cerebrovascular disease includes, but is not limited to, stroke.

Vascular disease includes, but is not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, and the like.

Edema includes, but is not limited to, peripheral tissue edema, hepatic congestion, splenic congestion, liver ascites, respiratory or lung congestion, and the like.

Insulinopathies include, but are not limited to, insulin resistance, Type I diabetes mellitus, Type II diabetes mellitus, glucose sensitivity, pre-diabetic state, syndrome X, and the like.

In one embodiment, a therapeutically effective combination of an epoxy steroidal compound (particularly eplerenone) and nicotinic acid or a nicotinic acid derivative is administered to a subject in need thereof to treat or prevent cardiovascular disorders selected from the group consisting of congenital disorders, valvular disorders, coronary artery disorders, nosocomial disorders, surgically-induced disorders, cardiomyopathic disorders, viral-induced disorders, bacterial-induced disorders, anatomic disorders, vascular disorders, transplantation-induced disorders, ischemic disorders, cardiac arrhythmia disorders, conduction disorders, thrombotic disorders, aortic disorders, coagulation disorders, connective tissue disorders, neuromuscular disorders, hematologic disorders, hypobaric disorders, endocrine disorders, pulmonary disorders, non-malignant tumor disorders, malignant tumor disorders and pregnancy-induced disorders. A group of cardiovascular disorders of interest comprises cardiovascular disorders selected from the group consisting of coronary artery disorders, cardiomyopathic disorders, aortic disorders, and connective tissue disorders. Another group of cardiovascular disorders of interest comprises cardiovascular disorders selected from the group consisting of congenital disorders, valvular disorders, nosocomial disorders, surgically-induced disorders, viral-induced disorders, bacterial-induced disorders, anatomic disorders, transplantation-induced disorders, conduction disorders, coagulation disorders, neuromuscular disorders, hematologic disorders, hypobaric disorders, endocrine disorders, pulmonary disorders, non-malignant tumor disorders, malignant tumor disorders and pregnancy-induced disorders.

Of particular interest are, for example, pathogenic conditions arising from atherosclerosis. Thus, in another embodiment the combination therapy of the present invention is used to prevent or treat myocardial infarction or stroke or endothelial dysfunction.

In another embodiment the combination therapy is used to prevent or treat a condition selected from the group consisting of hypertension, heart failure, left ventricular hypertrophy, sudden cardiac death and vascular disease.

In another embodiment the combination therapy is used to prevent or treat a condition selected from the group consisting of renal dysfunction and organ damage.

In another embodiment the combination therapy is used to prevent or treat a condition selected from the group consisting of diabetes, obesity, Syndrome X, cachexia and skin disorders.

In another embodiment the combination therapy is used to prevent or treat a condition selected from the group consisting of Alzheimer's Disease, dementia, depression, memory loss, drug addiction, drug withdrawal and brain damage.

In another embodiment the combination therapy is used to prevent or treat a condition selected from the group consisting of osteoporosis and muscle weakness.

In another embodiment the combination therapy is used to prevent or treat a condition selected from the group consisting of arthritis, tissue rejection, septic shock, anaphylaxis and tobacco-related pathological effects.

In another embodiment the combination therapy is used to prevent or treat pathological conditions that arise following coronary artery bypass graft (CABG) surgery.

In another embodiment the combination therapy is used to prevent or treat a condition selected from the group consisting of thrombosis and cardiac arrhythmias.

In another embodiment the combination therapy is used to prevent or treat a condition selected from the group consisting of tissue proliferative diseases and cancer.

In another embodiment the aldosterone receptor antagonist is used for the manufacture of a pharmaceutical composition for administration with nicotinic acid or a nicotinic acid derivative for the prevention or treatment of a pathogenic condition.

In another embodiment the aldosterone receptor antagonist is further combined with nicotinic acid or a nicotinic acid derivative for the manufacture of a pharmaceutical composition for the prevention or treatment of a pathogenic condition.

In the various embodiments of the invention, the aldosterone receptor antagonist used preferably is either spironolactone or an epoxy-steroidal compound. More preferably, the aldosterone receptor antagonist is eplerenone.

In addition, the combination therapies of the present invention are not limited to two components but may include one or more additional therapeutic compounds (e.g. a triple therapy) for treating the same or related disorders and providing some additional benefit to the patient.

In another embodiment of the combination therapy of the present invention, the aldosterone receptor antagonist and nicotinic acid or a nicotinic acid derivative are administered in combination with one or more additional compounds selected from the group consisting of angiotensin II receptor antagonists, angiotensin converting enzyme inhibitors, non-aldosterone antagonist-type diuretics, digoxin, calcium channel blockers, beta-adrenergic receptor blockers, COX-2 inhibitors, cholesterol synthesis inhibitors, non-steroidal anti-inflammatory compounds, alphal-adrenergic receptor antagonists and alpha2-adrenergic receptor agonists.

In addition to being particularly suitable for human use, the present combination therapy is also suitable for treatment of animals, including mammals such as horses, dogs, cats, rats, mice, sheep, pigs, and the like.

The novel combinations of the present invention exhibit, for example, improved efficacy, improved potency, and/or reduced dosing requirements for the active compounds relative to therapeutic regimens previously disclosed in the published literature.

Aldosterone Receptor Antagonists

The term “aldosterone antagonist ” or “aldosterone receptor antagonist” denotes a compound capable of binding to an aldosterone receptor, as a competitive inhibitor of the action of aldosterone itself at the receptor site, so as to modulate the receptor-mediated activity of aldosterone.

The aldosterone antagonists used in the methods of the present invention generally are spirolactone-type steroidal compounds. The term “spirolactone-type” is intended to characterize a structure comprising a lactone moiety attached to a steroid nucleus, typically at the steroid “D” ring, through a spiro bond configuration. A subclass of spirolactone-type aldosterone antagonist compounds consists of epoxy-steroidal aldosterone antagonist compounds such as eplerenone. Another subclass of spirolactone-type antagonist compounds consists of non-epoxy-steroidal aldosterone antagonist compounds such as spironolactone.

The epoxy-steroidal aldosterone antagonist compounds used in the method of the present invention generally have a steroidal nucleus substituted with an epoxy-type moiety. The term “epoxy-type” moiety is intended to embrace any moiety characterized in having an oxygen atom as a bridge between two carbon atoms, examples of which include the following moieties:

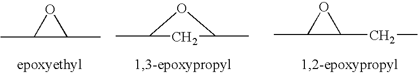

The term “steroidal”, as used in the phrase “epoxy-steroidal”, denotes a nucleus provided by a cyclopenteno-phenanthrene moiety, having the conventional “A”, “B”, “C” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto.

Epoxy-steroidal aldosterone antagonists suitable for use in the present methods include a family of compounds having an epoxy moiety fused to the “C” ring of the steroidal nucleus. Especially preferred are 20-spiroxane compounds characterized by the presence of a 9α,11α-substituted epoxy moiety. Compounds 1 through 11, below, are illustrative 9α,11α-epoxy-steroidal compounds that may be used in the present methods. A particular benefit of using epoxy-steroidal aldosterone antagonists, as exemplified by eplerenone, is the high selectivity of this group of aldosterone antagonists for the mineralocorticoid receptor. The superior selectivity of eplerenone results in a reduction in side effects, that can be caused by aldosterone antagonists that exhibit non-selective binding to non-mineralocorticoid receptors, such as androgen or progesterone receptors.

These epoxy steroids may be prepared by procedures described in Grob et al., U.S. Pat. No. 4,559,332. Additional processes for the preparation of 9,11-epoxy steroidal compounds and their salts are disclosed in Ng et al., WO97/21720 and Ng et al., WO98/25948.

TABLE 1
Aldosterone Receptor Antagonist

| Com- | |
|---|---|
| pound | |
| # | Structure and Name |
| 1 | 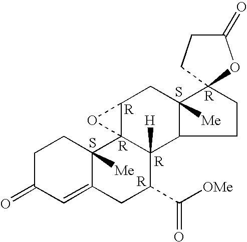 |
| | Pregn-4-ene-7,21-dicarboxylic acid, 9,11-epoxy- |
| | 17-hydroxy-3-oxo-,γ-lactone, methyl ester, |
| | (7α,11α,17β)- |
| 2 | 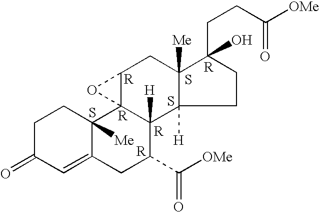 |
| | Pregn-4-ene-7,21-dicarboxylic acid, 9,11-epoxy- |
| | 17-hydroxy-3-oxo-,dimethyl ester, (7α,11α,17β)- |
| 3 | 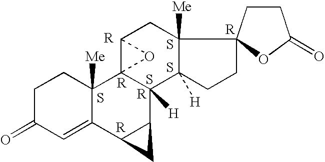 |
| | 3′H-cyclopropa[6,7]pregna-4,6-diene-21-carboxylic |
| | acid, 9,11-epoxy-6,7-dihydro-17-hydroxy-3-oxo-, |
| | γ-lactone, (6β, 7β, 11α, 17β)- |
| 4 | 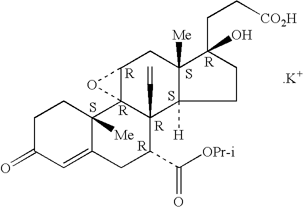 |
| | Pregn-4-ene-7,21-dicarboxylic acid,9,11-epoxy-17- |
| | hydroxy-3-oxo-,79(1-methylethyl) ester, |
| | monopotassium salt, (7α,11α,17β)- |
| 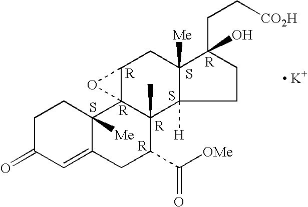 | |
| | Pregn-4-ene-7,21-dicarboxylic acid,9,11-epoxy-17- |
| | hydroxy-3-oxo-,7-methylethyl) ester,monopotassium |
| | salt, (7α,11α,17β)- |
| 6 | 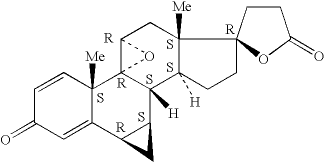 |
| | 3′H-cyclopropa[6,7]pregn-1,4,6-triene-21-carboxylic |
| | acid,9,11-epoxy-6,7-dihydro-17-hydroxy-3-oxo-, |
| | γ-lactone(6β,7β,11α)- |
| 7 | 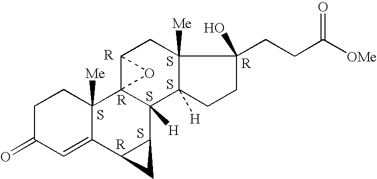 |
| | 3′H-cyclopropa[6,7]pregna-4,6-diene-21-carboxylic |
| | acid, 9,11-epoxy-6,7-dihydro-17-hydroxy-3-oxo-, |
| | methyl ester, (6β,7β,11α,17β)- |
| 8 | 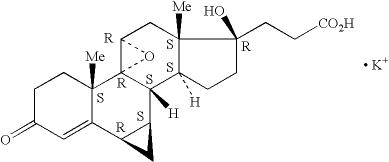 |
| | 3′H-cyclopropa[6,7]pregna-4,6-diene-21-carboxylic |
| | acid, 9,11-epoxy-6,7-dihydro-17-hydroxy-3-oxo-, |
| | monopotassium salt, (6β,7β,11α,17β)- |
| 9 | 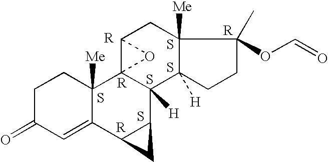 |
| | 3′H-cyclopropa[6,7]pregna-1,4,6-triene-21-carboxylic |
| | acid, 9,11-epoxy-6,7-dihydro-17-hydroxy-3-oxo-,γ- |
| | lactone(6β,7β,11α,17β)- |
| 10 | 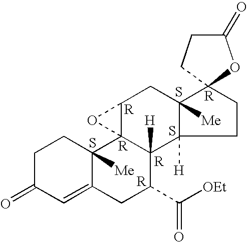 |
| | Pregn-4-ene-7,21-dicarboxylic acid, 9,11-epoxy- |
| | 17-hydroxy-3-oxo,γ-lactone, ethyl ester, |
| | (7α,11α,17β)- |
| 11 | 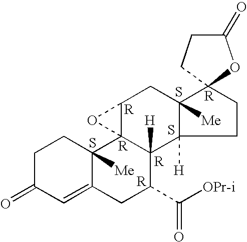 |
| | Pregn-4-ene-7,21-dicarboxylic acid, 9,11-epoxy- |
| | 17-hydroxy-3-oxo-,γ-lactone, 1-methylethyl |
| | ester (7α,11α,17β)- |

Of particular interest is the compound eplerenone (also known as: epoxymexrenone and CGP 30 083) which is compound 1 as shown above. The chemical name for eplerenone is pregn-4-ene-7,21-dicarboxylic acid, 9,11-epoxy-17-hydroxy-3-oxo, γ-lactone, methyl ester, (7α, 11α, 17α)-. This chemical name corresponds to the CAS registry name for eplerenone (the CAS registry number for eplerenone is 107724-20-9). U.S. Pat. No. 4,559,332 identifies eplerenone by the alternative name of 9α,11α-epoxy-7α-methoxycarbonyl-20-spirox-4-ene-3,21-dione. Such “spiroxane” nomenclature is further described, for example, at column 2, line 16 through column 4, line 48 of U.S. Pat. No. 4,559,332.

Eplerenone is an aldosterone receptor antagonist and has a higher specificity for aldosterone receptors than does, for example, spironolactone. Selection of eplerenone as the aldosterone antagonist in the present method would be beneficial to reduce certain side-effects such as gynecomastia that occur with use of aldosterone antagonists having less specificity.

Non-epoxy-steroidal aldosterone antagonists suitable for use in the present methods include a family of spirolactone-type compounds defined by Formula I:

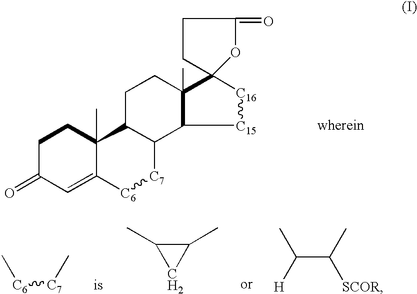

wherein R is lower alkyl of up to 5 carbon atoms, and

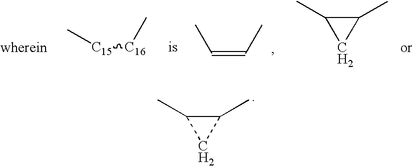

Lower alkyl residues include branched and unbranched groups, preferably methyl, ethyl and n-propyl.

Specific compounds of interest within Formula I are the following:

7α-acetylthio-3-oxo-4,15-androstadiene-[17(β-1′)-spiro-5′]perhydrofuran-2′-one;

3-oxo-7α-propionylthio-4,15-androstadiene-[17((β-1′)-spiro-5′]perhydrofuran-2′-one;

6β,7β-methylene-3-oxo4,15-androstadiene-[17((β-1′)-spiro-5 ′]perhydrofuran-2′-one;

15α,16α-methylene-3-oxo-4,7α-propionylthio-4-androstene[17(β-1′)-spiro-5′]perhydrofuran-2′-one;

6β,7β,15 α,16α-dimethylene-3-oxo-4-androstene[17(β-1′)-spiro-5′]-perhydrofuran-2′-one;

7α-acetylthio-15β,16β-Methylene-3-oxo-4-androstene-[17(β-1′)-spiro-5 ′]perhydrofuran-2′-one;

15β,16β-methylene-3-oxo-7β-propionylthio-4-androstene-[17(β-1′)-spiro-5′]perhydrofuran-2′-one; and

6β,7β,15β,16β-dimethylene-3-oxo-4-androstene-[17(β-1′)-spiro-5′]perhydrofuran-2′-one.

Methods to make compounds of Formula I are described in U.S. Pat. No. 4,129,564 to Wiechart et al. issued on Dec. 12, 1978.

Another family of non-epoxy-steroidal compounds of interest is defined by Formula II:

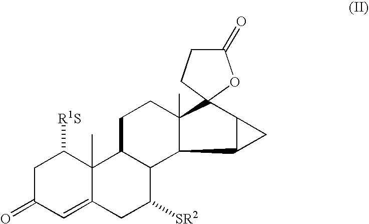

wherein R¹ is C₁₋₃-alkyl or C₁₋₃ acyl and R² is H or C₁₋₃-alkyl.

Specific compounds of interest within Formula II are the following:

1α-acetylthio-15β,16β-methylene-7α-methylthio-3-oxo-17α-pregn-4-ene-21,17-carbolactone; and

15β,16β-methylene-1α,7α-dimethylthio-3-oxo-17α-pregn-4-ene-21,17-carbolactone.

Methods to make the compounds of Formula II are described in U.S. Pat. No. 4,789,668 to Nickisch et al. which issued Dec 6, 1988.

Yet another family of non-epoxy-steroidal compounds of interest is defined by a structure of Formula III:

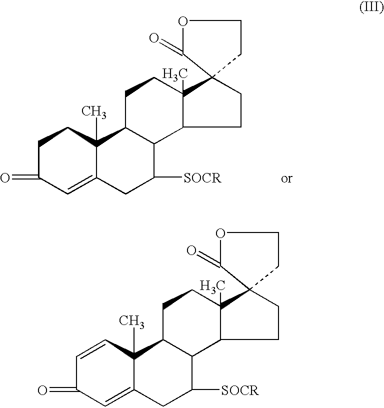

wherein R is lower alkyl, with preferred lower alkyl groups being methyl, ethyl, propyl and butyl. Specific compounds of interest include:

3β,21-dihydroxy-17α-pregna-5,15-diene-17-carboxylic acid γ-lactone;

3β,21-dihydroxy-17α-pregna-5,15-diene-17-carboxylic acid γ-lactone 3acetate;

3β,21-dihydroxy-17α-pregn-5-ene-17-carboxylic acid γ-lactone;

3β,21-dihydroxy-17α-pregn-5-ene-17-carboxylic acid γ-lactone 3acetate;

21-hydroxy-3-oxo-17α-pregn-4-ene-17-carboxylic acid γ-lactone;

21-hydroxy-3-oxo-17α-pregna-4,6-diene-17-carboxylic acid γ-lactone;

21-hydroxy-3-oxo-17α-pregna-1,4-diene-17-carboxylic acid γ-lactone;

7α-acylthio-21-hydroxy-3-oxo-17α-pregn-4-ene-17-carboxylic acid γlactone; and

7α-acetylthio-21-hydroxy-3-oxo-17α-pregn-4-ene-17-carboxylic acid γ-lactone.

Methods to make the compounds of Formula III are described in U.S. Pat. No. 3,257,390 to Patchett which issued Jun. 21, 1966.

Still another family of non-epoxy-steroidal compounds of interest is represented by Formula IV:

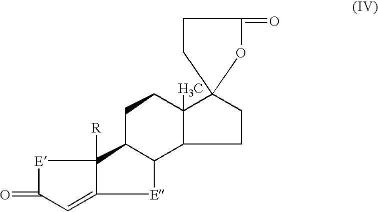

wherein E′ is selected from the group consisting of ethylene, vinylene and (lower alkanoyl)thioethylene radicals, E″ is selected from the group consisting of ethylene, vinylene, (lower alkanoyl)thioethylene and (lower alkanoyl)thiopropylene radicals; R is a methyl radical except when E′ and E″ are ethylene and (lower alkanoyl) thioethylene radicals, respectively, in which case R is selected from the group consisting of hydrogen and methyl radicals; and the selection of E′ and E″ is such that at least one (lower alkanoyl)thio radical is present.

A preferred family of non-epoxy-steroidal compounds within Formula IV is represented by Formula V:

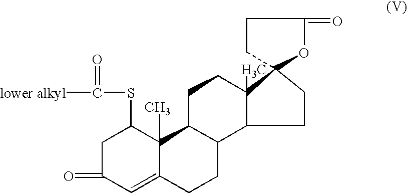

A more preferred compound of Formula V is 1-acetylthio-17α-(2-carboxyethyl)-17β-hydroxy-androst-4-en-3-one lactone.

Another preferred family of non-epoxy-steroidal compounds within Formula IV is represented by Formula VI:

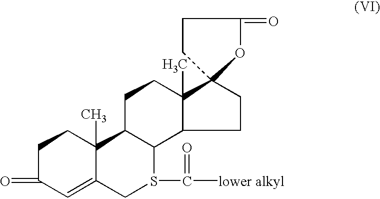

More preferred compounds within Formula VI include the following:

7α-acetylthio-17α-(2-carboxyethyl)-17β-hydroxy-androst-4-en-3-one lactone;

7β-acetylthio-17α-(2-carboxyethyl)-17β-hydroxy-androst-4-en-3-one lactone;

1α,7α-diacetylthio-17α-(2-carboxyethyl)-17β-hydroxy-androsta-4,6-dien-3-one lactone;

7α-acetylthio-17α-(2-carboxyethyl)-17β-hydroxy-androsta-1,4-dien-3-one lactone;

7α-acetylthio-17α-(2-carboxyethyl)-17β-hydroxy-19-norandrost-4-en-3-one lactone; and

7α-acetylthio-17α-(2-carboxyethyl)-17β-hydroxy-6α-methylandrost-4-en-3-one lactone;

In Formulae IV-VI, the term “alkyl” is intended to embrace linear and branched alkyl radicals containing one to about eight carbons. The term “(lower alkanoyl)thio” embraces radicals of the formula lower alkyl

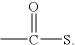

Of particular interest is the compound spironolactone having the following structure and formal name:

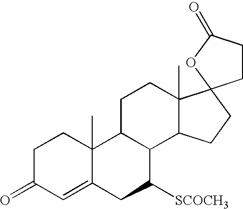

“spironolactone”: 17-hydroxy-7α-mercapto-3-oxo-17α-pregn-4-ene-21-carboxylic acid γ-lactone acetate.

Methods to make compounds of Formulae IV-VI are described in U.S. Pat. No. 3,013,012 to Cella et al. which issued Dec 12, 1961. Spironolactone is sold by Pharmacia Corporation under the trademark “ALDACTONE”, in tablet dosage form at doses of 25 mg, 50 mg and 100 mg per tablet. Spironolactone, in combination with hydrochlorothiazide, is sold by Pharmacia Corporation under the trademark “ALDACTAZIDE”, in tablet dosage form at spironolactone doses of 25 mg and 50 mg per tablet.

Another family of steroidal aldosterone antagonists is exemplified by drospirenone, [6R-(6alpha,7alpha,8beta,9alpha,10beta,13beta,14alpha,15alpha,16alpha, 17beta)]-1,3′,4′,6,7,8,9,10,11,12,13,14,15,16,20,21-hex adecahydro-10,13-dimethylspiro[17H-dicyclopropa[6,7:15,16]cyclopenta[a]phenanthrene-17,2′(5′H)-furan]-3,5′(2H)-dione, CAS registration number 67392-87-4. Methods to make and use drospirenone are described in patent GB 1550568 1979, priority DE 2652761 1976.

Nicotinic Acid Derivatives

Nicotinic acid derivatives useful in the combinations and methods of the present invention comprise a wide variety of structures and functionalities. In one embodiment the nicotinic acid derivatives used in the present invention are selected from Table 2. The therapeutic compounds of Table 2 can be used in the present invention in a variety of forms, including acid form, salt form, racemates, enantiomers, zwitterions, and tautomers. The individual patent documents referenced in Table 2 are each herein incorporated by reference.

TABLE 2

| Compound | | CAS Registry | Patent Document |
|---|---|---|---|
| Number | Common Name | Number | Reference |
| B-1 | Nicotinic Acid | 59-67-6 | |
| | (Niacin) | | |
| B-2 | Niceritrol | 5868-05-3 | GB 1022880 |
| B-3 | Acipimox | 51037-30-0 | GB 1351967 |
| B-4 | Acifran | 72420-38-3 | EP 0006305 |
| B-5 | Cyclohexylphenyl | 79781-87-6 | U.S. 4,321,268 & |
| | Nicotinate | | Drugs of the Future |
| | | | v.12, 349-351 |
| | | | (1987) |
| B-6 | Cyclohexylphenyl- | | U.S. 4,321,268 & |
| | oxide Nicotinate | | Drugs of the Future |
| | | | v.12, 349-351 |
| | | | (1987) |

In one embodiment, the nicotinic acid or nicotinic acid derivative is selected from the group consisting of niacin, niceritrol, acipimox, acifran, cyclohexylphenyl nicotinate, and cyclohexylphenyl-oxide nicotinate.

In another embodiment, the nicotinic acid derivative is niacin.

In another embodiment, the nicotinic acid derivative is niceritrol.

In another embodiment, the nicotinic acid derivative is acipimox.

In another embodiment, the nicotinic acid derivative is acifran.

In another embodiment, the nicotinic acid derivative is cyclohexylphenyl nicotinate.

In another embodiment, the nicotinic acid derivative is cyclohexylphenyl-oxide nicotinate.

In another embodiment, the nicotinic acid derivative is selected from the group consisting of niacin, niceritrol, acipimox, acifran, cyclohexylphenyl nicotinate, and cyclohexylphenyl-oxide nicotinate, and the aldosterone receptor antagonist is selected from the group consisting of eplerenone and spironolactone.

In another embodiment, the nicotinic acid derivative is niacin and the aldosterone receptor antagonist is eplerenone.

In another embodiment, the nicotinic acid derivative is niceritrol and the aldosterone receptor antagonist is eplerenone.

In another embodiment, the nicotinic acid derivative is acipimox and the aldosterone receptor antagonist is eplerenone.

In another embodiment, the nicotinic acid derivative is acifran and the aldosterone receptor antagonist is eplerenone.

In another embodiment, the nicotinic acid derivative is cyclohexylphenyl nicotinate and the aldosterone receptor antagonist is eplerenone.

In another embodiment, the nicotinic acid derivative is cyclohexylphenyl-oxide nicotinate and the aldosterone receptor antagonist is eplerenone.

In another embodiment, the nicotinic acid derivative is niacin and the aldosterone receptor antagonist is spironolactone.

In another embodiment, the nicotinic acid derivative is niceritrol and the aldosterone receptor antagonist is spironolactone.

In another embodiment, the nicotinic acid derivative is acipimox and the aldosterone receptor antagonist is spironolactone.

In another embodiment, the nicotinic acid derivative is acifran and the aldosterone receptor antagonist is spironolactone.

In another embodiment, the nicotinic acid derivative is cyclohexylphenyl nicotinate and the aldosterone receptor antagonist is spironolactone.

In another embodiment, the nicotinic acid derivative is cyclohexylphenyl-oxide nicotinate and the aldosterone receptor antagonist is spironolactone.

As noted above, the aldosterone receptor antagonists and compounds selected from the group consisting of nicotinic acid and nicotinic acid derivatives useful in the present combination therapy also may include the racemates and stereoisomers, such as diastereomers and enantiomers, of such agents. Such stereoisomers can be prepared and separated using conventional techniques, either by reacting enantiomeric starting materials, or by separating isomers of compounds of the present invention. Isomers may include geometric isomers, for example cis isomers or trans isomers across a double bond. All such isomers are contemplated among the compounds of the present invention. Such isomers may be used in either pure form or in admixture with those active agents described above. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

The compounds useful in the present invention as discussed below include their salts, solvates and prodrugs.

The compounds useful in the present invention also include tautomers.

Crystalline Forms of Active Compounds

Crystaline forms that are easily handled, reproducible in form, easily prepared, stable and which are non-hygroscopic have been identified for the aldosterone antagonist eplerenone. These include Form H, Form L, various crystalline solvates and amorphous eplerenone. These forms, methods to make these forms and use of these forms in preparing compositions and medicaments, are disclosed in the following publications, incorporated herein by reference: WO 01/41535 and WO 01/42272.

In one embodiment of the present invention, the aldosterone antagonist employed comprises Form L eplerenone.

In another embodiment of the present invention, the aldosterone antagonist employed comprises Form H eplerenone.

Definitions The term “combination therapy” means the administration of two or more therapeutic agents to treat a pathological condition. Such administration encompasses co-administration of these therapeutic agents in a substantially simultaneous manner, such as in a single capsule having a fixed ratio of active ingredients or in multiple, separate capsules for each active agent. In addition, such administration encompasses use of each type of therapeutic agent in a sequential manner. In either case, the treatment regimen will provide beneficial effects of the drug combination in treating the pathological condition.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prodrug” refers to a chemical compound that can be converted into a therapeutic compound by metabolic or simple chemical processes within the body of the subject.

The terms “prophylaxis” and “prevention” include either preventing the onset of a clinically evident pathological condition altogether or preventing the onset of a preclinically evident stage of a pathological condition in a subject. This term encompasses the prophylactic treatment of a subject at risk of developing a pathological condition.

The term “subject” as used herein refers to an animal, preferably a mammal, and particularly a human, who has been the object of treatment, observation or experiment.

The phrase “therapeutically-effective” qualifies the amount of each agent that will achieve the goal of improvement in pathological condition severity and the frequency of incidence over treatment of each agent by itself, especially while minimizing adverse side effects typically associated with alternative therapies.

The term “treatment” includes any process, action, application, therapy, procedure or the like, wherein a mammal, particularly a human, is subjected to medical aid with the object of improving the mammal's condition, directly or indirectly. Treatment also can include slowing or stopping the progression of a clinically evident cardiovascular condition altogether or slowing or stopping the progression of the onset of a preclinically evident stage of a cardiovascular condition in a subject.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl. The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like. The terms “alkenyl”, “lower alkenyl”, embrace radicals having “cis” and “trans” orientations, or alternatively, “E” and “Z” orientations. The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl. The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy. The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl. The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4- thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino. The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl. The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl. The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” denote NH₂O₂S—. The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl. The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and 5 the like and the aryl in said aroyl may be additionally substituted. The terms “carboxy” or “carboxyl”, whether used alone or with other terms, such as “carboxyalkyl”, denotes —CO2H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl. The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl. The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable. The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy. The term “aralkoxy” embraces aralkyl radicals attached through an oxygen atom to other radicals.

The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical. The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups which have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups which have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl. The term “aminocarbonyl” denotes an amide group of the formula —C(═O)NH₂. The term “alkylaminocarbonyl” denotes an aminocarbonyl group which has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above. The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

The compounds utilized in the methods of the present invention may be present in the form of free bases or pharmaceutically acceptable acid addition salts thereof. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of the present invention may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound.

Mechanism of Action Without being held to a specific mechanism of action for the present combination therapy, it is hypothesized that the administration of these selected aldosterone receptor antagonists and compounds selected from the group consisting of nicotinic acid and nicotinic acid derivatives in combination is effective because of the simultaneous and interrelated responses of tissues and/or organs to these two distinct classes of drugs: marked down-regulation of aldosterone-stimulated genetic effects in response to the aldosterone antagonist and reduced LDL cholesterol, and/or reduced triglyceridemia, and/or increased plasma HDL cholesterol, and/or increased HDL particles enriched in or exclusively containing apolipoprotein Al, and/or increased HDL particles reduced in or devoid of apolipoprotein All, and/or a decreased rate of HDL removal from plasma, in response to the nicotinic acid or nicotinic acid derivative. Such an effect would provide a cooperative benefit to the therapeutic use of an aldosterone receptor antagonist. Another mechanism for therapeutic interactions between an aldosterone antagonist and nicotinic acid or a nicotinic acid derivative could arise from anti-inflammatory effects of these drugs, in cooperation with reductions in serum LDL cholesterol, and/or triglycerides and hypertension, and increased HDL levels, and/or increased apolipoprotein AI levels, which would provide additional therapeutic benefit in treating or preventing atherosclerosis-related diseases.

Advantages of Combination Therapy The selected aldosterone receptor antagonists and compounds selected from the group consisting of nicotinic acid and nicotinic acid derivatives of the present invention act in combination to provide more than an additive benefit. For example, administration of a combination of an aldosterone receptor antagonist and a compound selected from the group consisting of nicotinic acid and nicotinic acid derivatives can result in the near-simultaneous reduction in pathogenic effects of multiple risk factors for atherosclerosis, such as high LDL cholesterol levels, high serum triglyceride levels, low HDL levels, low serum apolipoprotein Al levels, high aldosterone levels, high blood pressure, endothelial dysfunction, plaque formation and rupture, etc.

The methods of this invention also provide for the effective prophylaxis and/or treatment of pathological conditions with reduced side effects compared to conventional methods known in the art. For example, administration of nicotinic acid or nicotinic acid derivatives can result in side effects such as hepatotoxicity, elevated uric acid levels, reduced platelet counts, increased prothrombin times, reduced phosphorous levels, myotoxicity or myopathy, including rhabdomyolysis and related clinical sequelae. In addition, with nicotinic acid or most nicotinic acid derivatives a relatively large dose is required. Reduction of the dose of nicotinic acid or nicotinic acid derivative in the present combination therapy below conventional monotherapeutic doses will minimize, or even eliminate, the side-effect profile associated with the present combination therapy relative to the side-effect profiles associated with, for example, monotherapeutic administration of nicotinic acid or nicotinic acid derivatives. The reduction of the dose of nicotinic acid or nicotinic acid derivative in the present combination therapy below conventional monotherapeutic doses likewise will facilitate the administration of the nicotinic acid or nicotinic acid derivative to the subject relative to monotherapeutic administration of the nicotinic acid or nicotinic acid derivative.

Other benefits of the present combination therapy include, but are not limited to, the use of a selected group of aldosterone receptor antagonists that provide a relatively quick onset of therapeutic effect and a relatively long duration of action. For example, a single dose of one of the selected aldosterone receptor antagonists may stay associated with the aldosterone receptor in a manner that can provide a sustained blockade of mineralocorticoid receptor activation. Another benefit of the present combination therapy includes, but is not limited to, the use of a selected group of aldosterone receptor antagonists, such as the epoxy-steroidal aldosterone antagonists exemplified by eplerenone, which act as highly selective aldosterone antagonists, with reduced side effects that can be caused by aldosterone antagonists that exhibit non-selective binding to non-mineralocorticoid receptors, such as androgen or progesterone receptors. In addition, the use of an aldosterone antagonist may provide a direct benefit in preventing or treating liver dysfunction, including ascites formation and hepatic fibrosis.

Further benefits of the present combination therapy include, but are not limited to, the use of the methods of this invention to treat individuals who belong to one or more specific ethnic groups that are particularly responsive to the disclosed therapeutic regimens. Thus, for example, individuals of African or Asian ancestry may particularly benefit from the combination therapy of an aldosterone antagonist and nicotinic acid or a nicotinic acid derivative to treat or prevent a pathogenic condition.

Subject Populations Certain groups are more prone to disease modulating effects of aldosterone. Members of these groups that are sensitive to aldosterone are typically also salt sensitive, wherein individual's blood pressure will generally rise and fall with increased and decreased sodium consumption, respectively. While the present invention is not to be construed as limited in practice to these groups, it is contemplated that certain subject groups may be particularly suited for therapy with the present invention. Accordingly, subjects who can benefit from treatment or prophylaxis in accordance with the method of the present invention are human subjects generally exhibiting one or more of the following characteristics:

(a) the average daily intake of sodium chloride by the subject is at least about 4 grams, particularly where this condition is satisfied over any one month interval for at least one or more monthly intervals over a given annual period. The average daily intake of sodium by the subject preferably is at least about 6 grams, more preferably at least about 8 grams, and still more preferably at least about 12 grams.

(b) the subject exhibits an increase in systolic blood pressure and/or diastolic blood pressure of at least about 5%, preferably at least about 7%, and more preferably at least about 10%, when daily sodium chloride intake by the subject is increased from less than about 3 g/day to at least about 10 g/day.

(c) the activities ratio of plasma aldosterone (ng/dL) to plasma renin (ng/ml/hr) in the subject is greater than about 30, preferably greater than about 40, more preferably greater than about 50; and still more preferably greater than about 60.

(d) the subject has low plasma renin levels; for example, the morning plasma renin activity in the subject is less than about 1.0 ng/dL/hr, and/or the active renin value in the subject is less than about 15 pg/mL.

(e) the subject suffers from or is susceptible to elevated systolic and/or diastolic blood pressure. In general, the systolic blood pressure (measured, for example, by seated cuff mercury sphygmomanometer) of the subject is at least about 130 mm Hg, preferably at least about 140 mm Hg, and more preferably at least about about 150 mm Hg, and the diastolic blood pressure (measured, for example, by seated cuff mercury sphygmomanometer) of the subject is at least about 85 mm Hg, preferably at least about 90 mm Hg, and more preferably at least about 100 mm Hg.

(f) the urinary sodium to potassium ratio (mmol/mmol) of the subject is less than about 6, preferably less than about 5.5, more preferably less than about 5, and still more preferably less than about 4.5.

(g) the urinary sodium level of the subject is at least 60 mmol per day, particularly where this condition is satisfied over any one month interval for at least one or more monthly intervals over a given annual period. The urinary sodium level of the subject preferably is at least about 100 mmol per day, more preferably at least about 150 mmol per day, and still more preferably 200 mmol per day.

(h) the plasma concentration of one or more endothelins, particularly plasma immunoreactive ET-1, in the subject is elevated. Plasma concentration of ET-1 preferably is greater than about 2.0 pmol/L, more preferably greater than about 4.0 pmol/L, and still more preferably greater than about 8.0 pmol/L.

(i) the subject has blood pressure that is substantially refractory to treatment with an ACE inhibitor; particularly a subject whose blood pressure is lowered less than about 8 mm Hg, preferably less than 5 mm Hg, and more preferably less than 3 mm Hg, in response to 10 mg/day enalapril compared to the blood pressure of the subject on no antihypertensive therapy.

(j) the subject has blood volume-expanded hypertension or blood volume-expanded borderline hypertenision, that is, hypertension wherein increased blood volume as a result of increased sodium retension contributes to blood pressure.

(k) the subject is a non-modulating individual, that is, the individual demonstrates a blunted positive response in renal blood flow rate and/or in adrenal production of aldosterone to an elevation in sodium intake or to angiotensin II administration, particularly when the response is less than the response of individuals sampled from the general geographical population (for example, individuals sampled from the subject's country of origin or from a country of which the subject is a resident), preferably when the response is less than 40% of the mean of the population, more preferably less than 30%, and more preferably still less than 20%.

(I) the subject has or is susceptible to renal dysfunction, particularly renal dysfunction selected from one or more members of the group consisting of reduced glomerular filtration rate, microalbuminuria, and proteinuria.

(m) the subject has or is susceptible to cardiovascular disease, particularly cardiovascular disease selected from one or more members of the group consisting of heart failure, left ventricular diastolic dysfunction, hypertrophic cardiomyopathy, and diastolic heart failure.

(n) the subject has or is susceptible to liver disease, particularly liver cirrhosis.

(o) the subject has or is susceptible to edema, particularly edema selected from one or more members of the group consisting of peripheral tissue edema, hepatic or splenic congestion, liver ascites, and respiratory or lung congestion.

(p) the subject has or is susceptible to insulin resistance, particularly Type I or Type II diabetes mellitus, and/or glucose sensitivity.

(q) the subject is at least 55 years of age, preferably at least about 60 years of age, and more preferably at least about 65 years of age.

(r) the subject is, in whole or in part, a member of at least one ethnic group selected from the Asian (particularly from the Japanese) ethnic group, the American Indian ethnic group, and the Black ethnic group.

(s) the subject has one or more genetic markers associated with salt sensitivity.

(t) the subject is obese, preferably with greater than 25% body fat, more preferably with greater than 30% body fat, and even more preferably with greater than 35% body fat.

(u) the subject has one or more 1^{st}, 2^{nd}, or 3^{rd} degree relatives who are or were salt sensitive, wherein 1^{st} degree relatives means parents or relatives sharing one or more of the same parents, 2^{nd} degree relatives means grandparents and relatives sharing one or more of the same grandparents, and 3^{rd} degree relatives means great-grandparents and relatives sharing one or more of the same great-grandparents. Preferably, such individuals have four or more salt sensitive 1^{st}, 2_{nd}, or 3^{rd} degree relatives; more preferably, eight or more such relatives; even more preferably, 16 or more such relatives; and even more preferably still, 32 or more such relatives.

Unless otherwise indicated to the contrary, the values listed above preferably represent an average value, more preferably a daily average value based on at least two measurements.

Preferably, the subject in need of treatment satisfies at least two or more of the above-characteristics, or at least three or more of the above-characteristics, or at least four or more of the above-characteristics.

Dosages and Treatment Regimen

Aldosterone Receptor Antagonist Dosing

The amount of aldosterone receptor antagonist blocker that is administered and the dosage regimen for the methods of this invention depend on a variety of factors, including the age, weight, sex and medical condition of the subject, the severity of the pathogenic effect, the route and frequency of administration, and the particular aldosterone blocker employed, and thus may vary widely. A daily dose administered to a subject of about 0.001 to 30 mg/kg body weight, preferably between about 0.005 and about 20 mg/kg body weight, more preferably between about 0.01 and about 15 mg/kg body weight, still more preferably between about 0.05 and about 10 mg/kg body weight, and most preferably between about 0.01 to 5 mg/kg body weight, may be appropriate.

The daily dose of aldosterone antagonist administered to a human subject typically will range from about 0.1 mg to about 2000 mg. In one embodiment of the present invention, the daily dose range is from about 0.1 mg to about 400 mg. In another embodiment of the present invention, the daily dose range is from about 1 mg to about 200 mg. In a further embodiment of the present invention, the daily dose range is from about 1 mg to about 100 mg. In another embodiment of the present invention, the daily dose range is from about 10 mg to about 100 mg. In a further embodiment of the present invention, the daily dose range is from about 25 mg to about 100 mg. In another embodiment of the present invention, the daily dose is selected from the group consisting of about 5 mg, about 10 mg, about 12.5 mg, about 25 mg, about 50 mg, about 75 mg, and about 100 mg. In a further embodiment of the present invention, the daily dose is selected from the group consisting of about 25 mg, about 50 mg, and about 100 mg. A daily dose of aldosterone blocker that produces no substantial diuretic and/or anti-hypertensive effect in a subject is specifically embraced by the present method. The daily dose can be administered in one to four doses per day.

Dosing of the aldosterone antagonist can be determined and adjusted based on measurement of blood pressure or appropriate surrogate markers (including, without limitation, natriuretic peptides, endothelins, and other surrogate markers discussed below). Blood pressure and/or surrogate marker levels after administration of the aldosterone antagonist can be compared against the corresponding baseline levels prior to administration of the aldosterone antagonist to determine efficacy of the present method and titrated as needed. Non-limiting examples of surrogate markers useful in the method are surrogate markers for renal and cardiovascular disease.

Prophylactic Dosing

It is beneficial to administer the aldosterone antagonist prophylatically, prior to a diagnosis of said cardiovascular disorders, and to continue administration of the aldosterone antagonist during the period of time the subject is susceptible to the cardiovascular disorders. Individuals with no remarkable clinical presentation but that are nonetheless susceptible to pathologic effects therefore can be placed upon a prophylactic dose of an aldosterone antagonist compound. Such prophylactic doses of the aldosterone antagonist may, but need not, be lower than the doses used to treat the specific pathogenic effect of interest.

Cardiovascular Pathology Dosing

Dosing to treat pathologies of cardiovascular function can be determined and adjusted based on measurement of blood concentrations of natriuretic peptides. Natriuretic peptides are a group of structurally similar but genetically distinct peptides that have diverse actions in cardiovascular, renal, and endocrine homeostasis. Atrial natriuretic peptide (“ANP”) and brain natriuretic peptide (“BNP”) are of myocardial cell origin and C-type natriuretic peptide (“CNP”) is of endothelial origin. ANP and BNP bind to the natriuretic peptide-A receptor (“NPR-A”), which, via 3′,5′-cyclic guanosine monophosphate (cGMP), mediates natriuresis, vasodilation, renin inhibition, antimitogenesis, and lusitropic properties. Elevated natriuretic peptide levels in the blood, particularly blood BNP levels, generally are observed in subjects under conditions of blood volume expansion and after vascular injury such as acute myocardial infarction and remain elevated for an extended period of time after the infarction. (Uusimaa et al.: Int. J. Cardiol 1999; 69: 5-14).

A decrease in natriuretic peptide level relative to the baseline level measured prior to administration of the aldosterone antagonist indicates a decrease in the pathologic effect of aldosterone and therefore provides a correlation with inhibition of the pathologic effect. Blood levels of the desired natriuretic peptide level therefore can be compared against the corresponding baseline level prior to administration of the aldosterone antagonist to determine efficacy of the present method in treating the pathologic effect. Based upon such natriuretic peptide level measurements, dosing of the aldosterone antagonist can be adjusted to reduce the cardiovascular pathologic effect. Similarly, cardiac pathologies can also be identified, and the appropriate dosing determined, based on circulating and urinary cGMP Levels. An increased plasma level of cGMP parallels a fall in mean arterial pressure. Increased urinary excretion of cGMP is correlated with the natriuresis.

Cardiac pathologies also can be identified by a reduced ejection fraction or the presence of myocardial infarction or heart failure or left ventricular hypertrophy. Left ventricular hypertrophy can be identified by echo-cardiogram or magnetic resonance imaging and used to monitor the progress of the treatment and appropriateness of the dosing.

In another embodiment of the invention, therefore, the methods of the present invention can be used to reduce natriuretic peptide levels, particularly BNP levels, thereby also treating related cardiovascular pathologies.

Cardiovascular pathologies can also be identified by the presence of elevated blood or tissue levels of C-reactive protein (CRP).

In another embodiment of the invention, therefore, the methods of the present invention can be used to reduce C-reactive protein levels, thereby also treating related cardiovascular pathologies.

Renal Pathology Dosing

Dosing to treat pathologies of renal function can be determined and adjusted based on measurement of proteinuria, microalbuminuria, decreased glomerular filtration rate (GFR), or decreased creatinine clearance. Proteinuria is identified by the presence of greater than about 0.3 g of urinary protein in a 24 hour urine collection. Microalbuminuria is identified by an increase in assayable urinary albumin. Based upon such measurements, dosing of the aldosterone antagonist can be adjusted to ameliorate a renal pathologic effect.

Neuropathy Pathology Dosing Neuropathy, especially peripheral neuropathy, can be identified by and dosing adjustments based on, neurologic exam of sensory deficit or sensory motor ability.

Retinopathy Pathology Dosing

Retinopathy can be identified by, and dosing adjustments based on, opthamologic exam.

Nicotinic Acid Derivative Dosing

A total daily dose of nicotinic acid or a nicotinic acid derivative can generally be in the range of from about 500 to about 10,000 mg/day in single or divided doses, or about 1000 to about 8000 mg/day, or about 3000 to about 6000 mg/day in single or divided doses.

It is understood, however, that the specific dose level for each patient will depend upon a variety of factors including the activity of the specific agents employed, the age, body weight, general health, sex, diet, time of administration, rate of excretion, active agent combination selected, the severity of the particular conditions or disorder being treated, and the form of administration. Appropriate dosages can be determined in trials. The ratio of aldosterone receptor antagonist to nicotinic acid or nicotinic acid derivative (weight/weight), however, typically will range from about 1:1,000 to about 1:1, or about 1:500 to about 1:10, or about 1:200 to about 1:20, or about 1:100 to about 1:50.

The total daily dose of each drug can be administered to the patient in a single dose, or in proportionate multiple subdoses. Subdoses can be administered two to eight times per day. Doses can be in immediate release form or sustained release form effective to obtain desired results. Single dosage forms comprising the aldosterone receptor antagonist and nicotinic acid or nicotinic acid derivatives may be used where desirable.

Dosage Regimen

As noted above, the dosage regimen to prevent, treat, give relief from, or ameliorate a pathological condition, with the combinations and compositions of the present invention is selected in accordance with a variety of factors. These factors include the type, age, weight, sex, diet, and medical condition of the patient, the type and severity of the disease, the route of administration, pharmacological considerations such as the activity, efficacy, pharmacokinetics and toxicology profiles of the particular active agents employed, whether a drug delivery system is utilized, and whether the active agents are administered with other ingredients. Thus, the dosage regimen actually employed may vary widely and therefore deviate from the preferred dosage regimen set forth above.

Initial treatment of a patient suffering from a hyperlipidemic condition or disorder can begin with the dosages indicated above. Treatment generally should be continued as necessary over a period of several weeks to several months or years until the hyperlipidemic condition or disorder has been controlled or eliminated. Patients undergoing treatment with the combinations or compositions disclosed herein can be routinely monitored, for example in treating specific cardiovascular pathologies, by measuring blood pressure, ejection fraction, serum LDL or HDL or total cholesterol levels or total triglyceride levels by any of the methods well-known in the art, to determine the effectiveness of the combination therapy. Continuous analysis of such data permits modification of the treatment regimen during therapy so that optimal effective amounts of each type of active agent are administered at any time, and so that the duration of treatment can be determined as well. In this way, the treatment regimen/dosing schedule can be rationally modified over the course of therapy so that the lowest amount of aldosterone receptor antagonist and nicotinic acid or nicotinic acid derivative that together exhibit satisfactory effectiveness is administered, and so that administration is continued only so long as is necessary to successfully treat or prevent the pathological condition.

In combination therapy, administration of the aldosterone receptor antagonist and a compound selected from the group consisting of nicotinic acid and nicotinic acid derivatives may take place sequentially in separate formulations, or may be accomplished by simultaneous administration in a single formulation or separate formulations. Administration may be accomplished by any appropriate route, with oral administration being preferred. The dosage units used may with advantage contain one or more aldosterone receptor antagonists and one or more compounds selected from the group consisting of nicotinic acid and nicotinic acid derivatives in the amounts described above.

Dosing for oral administration may be with a regimen calling for a single daily dose, for multiple, spaced doses throughout the day, for a single dose every other day, for a single dose every several days, or other appropriate regimens. The aldosterone receptor antagonist and the nicotinic acid or nicotinic acid derivative used in the combination therapy may be administered simultaneously, either in a combined dosage form or in separate dosage forms intended for substantially simultaneous oral administration. The aldosterone receptor antagonists and the compounds selected from the group consisting of nicotinic acid and nicotinic acid derivatives also may be administered sequentially, with either agent being administered by a regimen calling for two-step ingestion. Thus, a regimen may call for sequential administration of the aldosterone receptor antagonist and the nicotinic acid or nicotinic acid derivative with spaced-apart ingestion of these separate, active agents. The time period between the multiple ingestion steps may range from a few minutes to several hours, depending upon the properties of each active agent such as potency, solubility, bioavailability, plasma half-life and kinetic profile of the agent, as well as depending upon the age and condition of the patient. Dose timing may also depend on the circadian or other rhythms for the pathological effects of agents, such as aldosterone, which may be optimally blocked at the time of their peak concentration. The combination therapy, whether administration is simultaneous, substantially simultaneous, or sequential, may involve a regimen calling for administration of the aldosterone receptor antagonist by oral or intravenous route and the nicotinic acid or nicotinic acid derivative by oral or intravenous route. Whether these active agents are administered by oral or intravenous route, separately or together, each such active agent will be contained in a suitable pharmaceutical formulation of pharmaceutically acceptable excipients, diluents or other formulations components. Examples of suitable pharmaceuticallyacceptable formulations are given above.

Combinations and Compositions

The present invention is further directed to combinations, including pharmaceutical compositions, comprising one or more aldosterone receptor antagonists and one or more compounds selected from the group consisting of nicotinic acid and nicotinic acid derivatives. In one embodiment, the present invention is directed to a combination comprising a first amount of the aldosterone receptor antagonist, or a pharmaceutically acceptable salt, ester, or prodrug thereof; a second amount of a compound selected from the group consisting of nicotinic acid and nicotinic acid derivatives, or a pharmaceutically acceptable salt, ester, conjugate acid, or prodrug thereof; and a pharmaceutically acceptable carrier. Preferably, the first and second amounts of the active agents together comprise a therapeutically effective amount of the agents. The preferred aldosterone receptor antagonists and compounds selected from the group consisting of nicotinic acid and nicotinic acid derivatives used in the preparation of the compositions are as previously set forth above. The combinations and compositions comprising an aldosterone receptor antagonist and nicotinic acid or a nicotinic acid derivative of the present invention can be administered for the prophylaxis and/or treatment of pathological conditions, as previously set forth, by any means that produce contact of these agents with their site of action in the body.

For the prophylaxis or treatment of the pathological conditions referred to above, the combination administered can comprise the active compounds per se. Alternatively, pharmaceutically acceptable salts are particularly suitable for medical applications because of their greater aqueous solubility relative to the parent compound.

The combinations of the present invention also can be presented with an acceptable carrier in the form of a pharmaceutical composition. The carrier must be acceptable in the sense of being compatible with the other ingredients of the composition and must not be deleterious to the recipient. The carrier can be a solid or a liquid, or both, and preferably is formulated with the compound as a unit-dose composition, for example, a tablet, which can contain from 0.05% to 95% by weight of the active compounds. Other pharmacologically active substances can also be present, including other compounds useful in the present invention. The pharmaceutical compositions of the invention can be prepared by any of the well-known techniques of pharmacy, such as admixing the components.

The combinations and compositions of the present invention can be administered by any conventional means available for use in conjunction with pharmaceuticals. Oral delivery of the aldosterone receptor antagonist and the nicotinic acid or nicotinic acid derivative is generally preferred. The amount of each active agent in the combination or composition that is required to achieve the desired biological effect will depend on a number of factors including those discussed below with respect to the treatment regimen.

Orally administrable unit dose formulations, such as tablets or capsules, can contain, for example, from about 0.1 to about 2000 mg, or about 0.5 mg to about 500 mg, or from about 0.75 to about 250 mg, or from about 1 to about 100 mg of the aldosterone receptor antagonist, and/or from about 50 to about 500 mg, or about 200 mg to about 1000 mg, or from about 500 to about 3000 mg, of the nicotinic acid or nicotinic acid derivative.

Oral delivery of the aldosterone receptor antagonist and the nicotinic acid or nicotinic acid derivatives of the present invention can include formulations, as are well known in the art, to provide immediate delivery or prolonged or sustained delivery of the drug to the gastrointestinal tract by any number of mechanisms. Immediate delivery formulations include, but are not limited to, oral solutions, oral suspensions, fast-dissolving tablets or capsules, disintegrating tablets and the like. Prolonged or sustained delivery formulations include, but are not limited to, pH sensitive release from the dosage form based on the changing pH of the gastrointestinal tract, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester. Non-limiting examples of formulations, including extended release formulations, as found in NIASPAN® tablets (Kos Pharmaceuticals), are disclosed in U.S. Pat. No. 6,080,428 and U.S. Pat. No. 6,129,930, both incorporated herein by reference.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active agent(s) and the carrier (which can constitute one or more accessory ingredients). In general, the compositions are prepared by uniformly and intimately admixing the active agent(s) with a liquid or finely divided solid carrier, or both, and then, if necessary, shaping the product. For example, a tablet can be prepared by compressing or molding a powder or granules of the active agents, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

In any case, the amount of aldosterone receptor antagonist and compound selected from the group consisting of nicotinic acid and nicotinic acid derivatives that can be combined with carrier materials to produce a single dosage form to be administered will vary depending upon the host treated and the particular mode of administration. The solid dosage forms for oral administration including capsules, tablets, pills, powders, and granules noted above comprise the active agents of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Pharmaceutically acceptable carriers encompass all the foregoing and the like. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa. , 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

Pharmaceutical combinations suitable for use in the present invention are described in Table 3.

TABLE 3
EXAMPLES OF COMBINATIONS

| ALDOSTERONE | NICOTINIC ACID OR NICOTINIC ACID |
|---|---|
| RECEPTOR | DERIVATIVES |
| ANTAGONIST | (COMPOUND NUMBER - TABLE 2) |
| Eplerenone | B-1 |
| Eplerenone | B-2 |
| Eplerenone | B-3 |
| Eplerenone | B-4 |
| Eplerenone | B-5 |
| Eplerenone | B-6 |
| Spironolactone | B-1 |
| Spironolactone | B-2 |
| Spironolactone | B-3 |
| Spironolactone | B-4 |
| Spironolactone | B-5 |
| Spironolactone | B-6 |

For therapeutic purposes, the active components of this combination therapy invention are ordinarily combined with one or more adjuvants appropriate to the indicated route of administration. If administered per os, the components may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The components may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Kits

The present invention further comprises kits that are suitable for use in performing the methods of treatment and/or prophylaxis described above. In one embodiment, the kit contains a first dosage form comprising one or more aldosterone receptor antagonists and a second dosage form comprising one or more compounds selected from the group consisting of nicotinic acid and nicotinic acid derivatives in quantities sufficient to carry out the methods of the present invention. Preferably, the first dosage form and the second dosage form together comprise a therapeutically effective amount of these agents for the prophylaxis and/or treatment of a pathological condition.

In one embodiment, the kit contains a first dosage form comprising eplerenone or spironolactone and a second dosage form comprising nicotinic acid or a nicotinic acid derivative identified in Table 2 in quantities sufficient to carry out the methods of the present invention.

In another embodiment, the kit contains a first dosage form comprising the eplerenone and a second dosage form comprising nicotinic acid or a nicotinic acid derivative.

In another embodiment, the kit contains a first dosage form comprising the eplerenone and a second dosage form comprising nicotinic acid or a nicotinic acid derivative identified in Table 2.

In another embodiment, the kit contains a first dosage form comprising the eplerenone and a second dosage form comprising nicotinic acid or a nicotinic acid derivative selected from the group consisting of niacin, acipimox, niceritrol and acifran.

In another embodiment, the kit contains a first dosage form comprising the spironolactone and a second dosage form comprising nicotinic acid or a nicotinic acid derivative.

In another embodiment, the kit contains a first dosage form comprising the spironolactone and a second dosage form comprising nicotinic acid or a nicotinic acid derivative identified in Table 2.

In another embodiment, the kit contains a first dosage form comprising spironolactone and a second dosage form comprising nicotinic acid or a nicotinic acid derivative selected from the group consisting of acipimox, niceritrol and acifran.

In another embodiment, the kit further comprises written instructions stating how the contents of the kit can be used by the subject. The written instructions will be useful, for example, for the subject to obtain a therapeutic effect without inducing unwanted side-effects. In another embodiment the written instructions comprise all or a part of the product label approved by a drug regulatory agency for the kit.

The following nonlimiting examples serve to illustrate various aspects of the present invention.

### EXAMPLE 1

Therapeutic Treatment

Non-limiting examples of in vitro and in vivo testing schemes and protocols that 5 can be used to evaluate the therapeutic benefit of (1) aldosterone receptor antagonists and (2) nicotinic acid or nicotinic acid derivatives, either separately or in combination, for treating or preventing pathogenic conditions are described in references listed below, which are incorporated herein by reference:

| | PATHOGENIC |
|---|---|
| REFERENCE | CONDITIONS |
| (1) WO 02/09683; | Inflammation |
| (2) WO 01/95893; | Hypertension, heart failure |
| (3) WO 01/34132; | Restenosis |
| (4) WO 00/69446; | Hypercholesterolemia, |
| | atherosclerosis |
| (5) WO 00/69445; | Hypercholesterolemia, |
| | atherosclerosis |
| (6) WO 00/51642; | Circulatory disorders, |
| | hypertension, |
| | heart failure |
| (7) WO 00/45818; | Diabetes |
| (8) WO 00/45817; | Hyperlipidemia, |
| | atherosclerosis |
| (9) WO 99/66930; | Hypercholesterolemia |
| (10) WO 99/11260; | Hypertension, |
| | hyperlipidemia, |
| | atherosclerosis |
| (11) U.S. Pat. No. 6,180,597; | Endothelial dysfunction |
| (12) U.S. Pat. No. 5,932,587; | Dyslipidemia, atherosclerosis |
| (13) U.S. Pat. No. 5,730,992; | Skin disorders |
| (14) Pitt, et al. NEJM 341, 709-717 | Heart failure |
| (1999); | |
| (15) Pitt, et al. Cardiovasc Drug Ther | Heart failure |
| 15: 79-87 (2001); | |
| (16) Blazer-Yost, et al. Am. J. Physiol | Aldosterone stimulated |
| 272, C1928-C1935 (1997); | sodium transport |
| (18) Vijan, et al. J Gen Intern Med 12, | Microvascular disease, |
| 567-580 (1997); | atherosclerosis, diabetes |
| (19) Gentile, et al. Diabetes, Obesity | Diabetes, |
| and Metabolism 2, 355-362 (2000); | hypercholesterolemia |
| (20) Sheng-Fang, et al. Am J Cardiol 86, | Left ventricular hypertrophy |
| 514-518 (2000); | |
| (21) Jick, et al. Lancet 356, 1627-1631 | Dementia |
| (2000); | |
| (22) Albert, et al. JAMA 286, 64-70 | C-reactive protein, |
| (2001); | inflammation |
| (23) Ridker, et al. NEJM 344, 1959-1965 | C-reactive protein, |
| (2001); | inflammation |
| (24) Wang, et al. JAMA 283, 3211-3216 | Bone disorders |
| (2000); | |
| (25) Meier, et al. JAMA 283, 3205-3210 | Bone disorders |
| (2000); | |
| (26) Sugiyama, et al. Biochem Biophys | Osteoporosis |
| Res Commun 271, 688-692 (2000); | |
| (27) Mundy, et al. Science 286, 1946- | Osteoporosis |
| 1949 (1999); and | |
| (28) Xiao, et al. J Endocrinol 165, 533- | Cell proliferation |
| 536 (2000). | |

### EXAMPLE 2

Therapeutic Treatment to Improve Endothelial Dysfunction in Diet Induced Atherosclerosis in Rabbits

A study is conducted to test the efficacy of a therapeutic combination of an aldosterone receptor antagonist and nicotinic acid or a nicotinic acid derivative to determine if the combination therapy can improve or prevent the endothelial dysfunction that occurs with atherosclerosis.

Methods: New Zealand white rabbits are randomized to four treatment groups. 32 Rabbits are placed on normal (NC) or 1% cholesterol chow (HC) for 8 weeks. After the first 2 weeks 16 rabbits are randomized to receive either saline (S) or the aldosterone receptor antagonist eplerenone (20 mg/kg twice daily, by gavage) plus niacin (50 mg/kg twice daily, by gavage) for an additional 6 weeks. Rabbits are euthanized at the end of 8 weeks and the aortas extracted for isometric tension studies and estimation of superoxide (O₂⁻) generation in vessel segments by lucigenin chemiluminescence (250 μM). Vessels are preconstricted with phenylephrine (3×10⁻⁷) to approximately 50% of peak constriction and dose responses to acetylcholine (Ach) and nitroglycerin (NTG) tested.

Results: The peak relaxations to Ach, NTG, ED₅₀ (M) values and O₂⁻ counts (per mg of dry weight) are determined. It is expected that combination therapy will improve endothelial function and reduce O₂⁻ generation in diet induced atherosclerosis.

In another aspect of this Example, a therapeutic benefit may also be obtained with other combinations using a different or additional aldosterone receptor antagonist, such as spironolactone, and/or a different or additional nicotinic acid derivative, such as acipimox, niceritrol or acifran. EXAMPLE 3

Comparison Study of the Efficacy and Safety of Eplerenone and Niacin, Both Alone and in Combination with Each other in Patients with Left Ventricular Hypertrophy and Essential Hypertension.

A clinical study is conducted to evaluate the effect of niacin and eplerenone, given alone and in combination with each other, following nine months of treatment on change in blood pressure (BP) and on change in left ventricular mass (LVM) as measured by magnetic resonance imaging (MRI) in patients with left ventricular hypertrophy (LVH) and with essential hypertension. The study is a multicenter, randomized, double-blind, placebo run-in, parallel group trial involving a minimum of 150 completed patients with LVH and essential hypertension and consisting of a one- to two-week pretreatment screening period followed by a two-week single-blind placebo run-in period and a nine-month double-blind treatment period.

Patients who will enter the single-blind placebo run-in period (1) will have a prior electrocardiogram that shows LVH (a) by the Sokolow Lyon voltage criteria (Sokolow M et al. Am Heart J 1949;37:161), or (b) by the Devereux criteria (LVMI =134 g/m² for males and =110 g/M² for females; see Neaton JD et al. JAMA 1993;27:713-724); and (2) will have a seated blood pressure that as follows: (a) seDBP <110 mmHg and seSBP =180 mmHg if currently treated with antihypertensive medication, or (b) seDBP =85 mmHg and <114 mmHg and seSBP >140 mmHg and =200 mmHg if not currently treated with antihypertensive medication.

During the single-blind placebo run-in period at Visit 2, all patients must have an echocardiogram that demonstrates LVH per the Devereux criteria. After completing the two-week single-blind placebo run-in period, and after an MRI has been received, and approved as acceptable by the core laboratory, patients will be randomized to one of three groups: eplerenone, niacin, or eplerenone plus niacin. For the first two weeks of double-blind treatment patients will receive (1) eplerenone 50 mg plus placebo, (2) niacin 500 mg plus placebo, or (3) eplerenone 50 mg plus niacin 500 mg. The dose of study medication will be force-titrated for all patients at Week 2 to (1) eplerenone 100 mg plus placebo, (2) niacin 1000 mg plus placebo, or (3) eplerenone 100 mg plus niacin 1000 mg. At Week 4 the dose of study medication will be force-titrated for all patients to (1) eplerenone 200 mg plus placebo, (1) niacin 2000 mg plus placebo, or (3) eplerenone 200 mg plus niacin 2000 mg. If at Week 16 or at any subsequent visit, the patient exhibits sustained uncontrolled DBP (i.e., seDBP =90 mmHg or seSBP >180 mmHg which persists at two consecutive visits, 3-10 days apart), the patient will be withdrawn from study participation. Patients will also be withdrawn from study participation if they develop signs of hepatotoxicity or display symptomatic rhabdomyolysis.

If a patient is taking double-blind treatment alone and experiences symptomatic hypotension at any time during the trial, the patient will be withdrawn. Those patients taking open-label medications will have the open-label medications down-titrated in the reverse sequence as they were added until hypotension is resolved. If after all open-label medications are discontinued symptomatic hypotension is still present, the patient will be withdrawn from the trial. At any time during the study, if serum potassium level is elevated (>5.5 mEq/L) on repeat measurement (with BUN and creatinine levels drawn as well, sample split and sent to local and central laboratories, treatment decision based on local value) at two consecutive visits 1-3 days apart, the patient will be withdrawn. NOTE: If BUN and/or creatinine levels are significantly elevated over baseline (creatinine =2.0 mg/dL or =1.5× baseline value or BUN =35 mg/dL or =2× baseline 5 value), the patient should be followed under medical treatment until resolved.

Patients will return to the clinic for evaluations at Weeks 0, 2, 4, 6, 8, 10, 12, 16, and monthly thereafter for a total of nine months. Heart rate, BP, serum potassium levels, plasma lipid and lipoprotein levels and adverse events will be assessed at each visit. BUN and creatinine levels will be determined at Weeks 2 and 6. Additional laboratory assessments of blood for clinical safety will be done monthly. Routine urinalysis will be done every three months. A neurohormone profile (plasma renin [total and active], serum aldosterone, and plasma cortisol) and special studies (PIIINP, PAI, microalbuminuria, and tPA) will be done at Weeks 0, 12, and at Months 6 and 9. A blood sample for genotyping will be collected at Week 0. At screening and at Month 9, a 12-lead ECG and physical examination will be done. An MRI to assess changes in LV mass, a blood sample for storage retention, a blood sample for thyroid stimulating hormone (TSH), and a 24-hour urine collection for albumin, potassium, sodium, and creatinine will be done at Week 0 and at Month 9. A 24-hour urine collection for urinary aldosterone will be done at Weeks 0, 12 and at Months 6 and 9. In case of early termination, an MRI and blood sample for TSH will be done for those patients who have received double-blind treatment for at least three months. At Weeks 0, 12 and Months 6 and 9, pharmacoeconomic data will be collected on all patients.

The primary measure of efficacy is the change from baseline in LVM, as assessed by MRI. Secondary measures of efficacy will be the following: (1) the change from baseline in LVM among the three treatment groups; (2) the change from baseline of seated trough cuff DBP (seDBP) and SBP (seSBP) in each of the three treatment groups; (3) aortic compliance and ventricular filling parameters; (4) plasma lipid and lipoprotein levels and (5) special studies (PIIINP, microalbuminuria, PAI, and tPA). Additionally, the long-term safety and tolerability of the three treatment groups will be compared.

The primary objective of the study is to compare effects of the different therapies on changes in left ventricular mass (LVM) in patients with LVH and with essential hypertension. The secondary objectives of the study are the following: (1) to compare the change from baseline in LVM among the three treatment groups; (2) to compare the antihypertensive effect among the three treatment groups as measured by seated trough cuff DBP and SBP; (3) to compare the effect of the three treatment groups on aortic compliance and ventricular filling parameters as measured by MRI; (4) to compare the effect of the three treatment groups on plasma markers of fibrosis by measuring the aminoterminal propeptide of Type III procollagen (PIIINP), on renal glomerular function by measuring microalbuminuria, and on fibrinolytic balance by measuring plasminogen activator inhibitor (PAI) and tissue plasminogen activator (tPA); (5) to compare the effect of the three treatment groups on plasma lipid and lipoprotein levels; and (6) to compare the long-term safety and tolerability of the three treatment groups.

Subgroup analyses of the primary and secondary efficacy measures can be performed with respect to other subgroups based on, for example, baseline recordings of such factors as sex, age, plasma renin levels, aldosterone/renin activities ratio, urinary sodium to potassium ratio, presence of diabetes, history of hypertension, history of heart failure, history of renal dysfunction, dyslipidemia, and the like. Subgroups based on continuous measures such as age can be dichotomized at the median value.

In another aspect of this Example, a therapeutic benefit may also be obtained with other combinations using a different or additional aldosterone receptor antagonist, such as spironolactone, and/or a different or additional nicotinic acid derivative, such as acipimox, niceritrol or acifran. EXAMPLE 4

Therapy to Prevent or Treat Endothelial Dysfunction in Humans.

Patients, at risk for or suffering from cardiovascular disease, are divided into 2 groups: (1) Treated, receiving 50 mg of the aldosterone receptor antagonist eplerenone and 500 mg of niacin for 2 months, or (2) Placebo for 2 months. At intervals of 2 weeks, starting 1 month prior to treatment, patients will be tested for endothelial function as follows: After 20 minutes of supine rest, the nondominant brachial artery is cannulated under local anesthesia. After 30 minutes of saline infusion, baseline forearm blood flow is measured by forearm venous-occlusion plethysmography. Drugs are then infused into the study arm with a constant rate infuser. Forearm blood flow is measured at each baseline and during the last two minutes of each drug infusion. Blood pressure is measured in the non-infused (control) arm at regular intervals throughout the study.

Drug Infusions

First, acetylcholine (endothelium-dependant vasodilator) is infused at 25, 50, and 100 mmol/minute, each for five minutes. This is followed by sodium nitroprusside (endothelium independent vasodilator) at 4.2, 12.6, and 37.8 nmol/min, each for 5 minutes, and then N-monoethyl-L-arginine (L-NMMA; competitive NO synthase inhibitor) at 1, 2, and 4 μmol/min for 5 minutes each. This is followed by angiotensin I (vasoconstrictor only through conversion to angiotensin II) at 64, 256, and 1024 pmol/min for 7 minutes each. Between the different drugs, the drug infusion is flushed with saline for 20 to 30 minutes to allow sufficient time for the forearm blood flow to return to baseline values

Results

It is expected that treatment with the combination of eplerenone and niacin will significantly increase the forearm blood flow response to acetylcholine (percentage change in forearm blood flow), with an associated increase in vasoconstriction due to L-NMMA.

In another aspect of this Example, a therapeutic benefit may also be obtained with other combinations using a different or additional aldosterone receptor antagonist, such as spironolactone, and/or a different or additional nicotinic acid derivative, such as acipimox, niceritrol or acifran.

### EXAMPLE 5

A Double-Blind Study to Assess the Change in Coronary Artery Atheroma Post Cardiac Transplantation as Measured by Ivus after 12 Months Dosing.

Objectives: The primary objective of the study is to measure change in maximal mean intimal thickness of the anterior descending coronary artery as assessed by intravascular ultrasonography (IVUS) (read centrally) after 12 months of treatment with combination therapy of the aldosterone receptor antagonist eplerenone and niacin. A change from baseline of 30% in intimal thickness is considered clinically significant. The secondary objectives of the study are to measure the effects on coronary artery atheroma and to compare effects of the combination therapy with the following assessments:

evidence of organ rejection as assessed by adverse event reports.

measurement of LDL-C, HDL-C, apoB, apoA-1, Lp (a) concentrations, ex vivo platelet aggregation, fibrinogen, and the concentrations of circulating markers of vascular inflammation.

comparison of plasma lipid and lipoprotein values after 52 weeks of treatment.

measurement of inflammatory markers after 52 weeks of treatment (HLA antigen VCAM/ICAM expression as assessed by biopsy).

to determine the drug's safety and tolerability.

Type and number of subjects: Approximately 40 men and women (aged 18 years and older) post cardiac transplant with hypercholesterolemia and triglycerides <400 mg/dl at the time of randomization.

Trial treatment: Once daily doses of eplerenone (50 mg) or niacin (500 mg) for two weeks, then titration of dose to 100 mg of eplerenone and 1000 mg niacin. Patients who have had their dose titrated up may have their dose titrated down, at the discretion of the investigator.

Duration of treatment: Eligible subjects randomized to 1 of 2 treatment groups, standard care plus combination therapy or standard care plus placebo, for 52 weeks. Primary measure: Mean change from baseline in maximal mean intimal thickness, as assessed by IVUS (read centrally).

Secondary measures: Percent change from baseline in LDL-C at 6 and 12 months.

Percent change from baseline in total cholesterol (TC), low-density lipoprotein cholesterol (LDL-C), high-density lipoprotein cholesterol (HDL-C), LDL-C/HDL-C, TC/HDL-C, non-HDL-C/HDL-C, and triglycerides (TG). Percent change from baseline in ApoB, ApoB/ApoA-1, ApoA-1, ApoA-2, ApoA-1/ApoA-2, Lp (a), and particle subfractions at 6 and 12 months. Percentage of subjects on each of the possible titrated doses at 12 months. Endocardial rejection will be considered an adverse event. Percent change from baseline in inflammatory markers (HLA antigen level and ICAM/VCAM expression). Safety evaluation as determined by adverse events, physical examination, and laboratory data.

Trial Design: This is a multicenter, randomized, double-blind clinical trial. Within 1 to 4 weeks post surgery, subjects are randomized to receive either the combination Therapy or placebo for 52weeks. Subjects must not have received any other lipid lowering therapy post-surgery.

Inclusion Criteria: (1) have undergone cardiac transplantation up to four weeks prior to randomization (2) fasting TG concentrations of <4.52 mmol/L (400 mg/dl) Exclusion criteria Any of the following is regarded as a criterion for exclusion from the trial: (1) Use of other cholesterol lowering drugs or lipid lowering dietary supplements or food additives post-transplantation prior to entering the study; (2) history of serious or hypersensitivity reactions to aldosterone receptor antagonists or bile acid sequestering resins; (3) participation in another investigational drug trial less than 4 weeks before randomization into this trial; (4) subjects randomized to double-blind treatment who subsequently withdrew cannot re-enter this trial; (5) serious or unstable medical or psychological conditions that, in the opinion of the investigator, would compromise the subject's safety or successful participation in the trial.

In another aspect of this Example, a therapeutic benefit may also be obtained with other combinations using a different or additional aldosterone receptor antagonist, such as spironolactone, and/or a different or additional nicotinic acid derivative, such as acipimox, niceritrol or acifran.

### EXAMPLE 6

Evaluation of Combination Therapy in Cholesterol-Fed Rabbits.

A study is conducted to test the efficacy of a therapeutic combination of the aldosterone receptor antagonist eplerenone and niacin to determine if the combination therapy can improve or prevent atherosclerosis in cholesterol-fed rabbits.

Methods: Groups of male, New Zealand white rabbits are placed on a standard diet (100 g/d) supplemented with 0.3% cholesterol and 2% corn oil (Ziegler Brothers, Inc., Gardners, PA). Water is available ad lib. At the start of the diet half of the animals receive either 20 mg/kg per day eplerenone and 100 mg/kg per day gemfibrozil. The remaining rabbits serve as untreated controls. Groups of controlled and treated animals are killed after 1 and 3 months of treatment. Tissues are removed for characterization of atherosclerotic lesions. Blood samples are taken for determination of plasma lipid and lipoprotein concentrations. Mean arterial pressure is measured in conscious animals at the end of the study.

Plasma lipids. Plasma for lipid analysis is obtained by withdrawing blood from the ear vein into EDTA-containing tubes (Vacutainer; Becton Dickinson & Co., Rutherford, N.J.), followed by centrifugal separation of the cells. Total cholesterol is determined enzymatically, using the cholesterol oxidase reaction. HDL cholesterol is also measured enzymatically, after selective precipitation of LDL and VLDL by dextran sulfate with magnesium. Plasma triglyceride levels are determined by measuring the amount of glycerol released by lipoprotein lipase through an enzyme-linked assay.

Blood Pressure. On the day blood pressure is measured, animals are orally dosed in the morning as usual. Catheters for blood pressure are then implanted in animals anesthetized with ketamine/xylazine mixture. Measurements are begun after 4 h of recovery, ˜5 h after oral dosing. Resting mean arterial pressure is measured in conscious rabbits with a pressure transducer (Statham Instruments, Inc., Oxnard, Calif.) connected to a catheter introduced through the right carotid artery and positioned into the ascending aorta. Multiple injections of peptides of increasing concentrations are made at intervals of 5-10 min after blood pressure returns to baseline. The duration of drug effects on the pressor response to peptide injections is measured in conscious, catheterized animals at intervals ranging from 0.5 to 24 h after a single oral dose.

Atherosclerosis. Animals are killed by pentobarbital injection. Thoracic aortas are rapidly removed, immersion fixed in 10% neutral buffered Formalin, and stained with oil red 0 (0.3%). After a single longitudinal incision along the wall opposite the arterial ostia, the vessels are pinned open for evaluation of the plaque area. The percent plaque coverage is determined from the values for the total area examined and the stained area, by threshold analysis using a true color image analyzer (Videometric 150; American Innovasion, Inc., San Diego, Calif.) interfaced to a color camera (Toshiba 3CCD) mounted on a dissecting microscope. Tissue cholesterol is measured enzymatically as described, after extraction with a chloroform/methanol mixture (2:1).

In vitro vascular response. The abdominal aortas are rapidly excised, after injection of sodium pentobarbital, and placed in oxygenated Krebs-bicarbonate buffer. After removal of perivascular tissue, 3-mm ring segments are cut, placed in a 37° C. muscle bath containing Krebs-bicarbonate solution, and suspended between two stainless steel wires, one of which is attached to a force transducer (Grass Instrument Co., Quincy, Mass.). Force changes in response to angiotensin II added to the bath are recorded on a chart recorder (model 8, Grass Instrument Co.).

Results

The primary measure of efficacy is a decrease in the amount of lipid stained aortic area for the treated group, relative to the control group. Secondary measures of efficacy include improvement in the in vitro vascular response (a measure of endothelial dysfunction) for the treated group, relative to the control group. In addition, a decrease in blood pressure, and improved plasma lipid and lipoprotein profiles for the treated group, relative to the control group, are also predictive of efficacy for combination therapy. Safety and tolerability of the drug combination will also provide data useful in evaluating this therapy.

In another aspect of this Example, a therapeutic benefit may also be obtained with other combinations using a different or additional aldosterone receptor antagonist, such as spironolactone, and/or a different or additional nicotinic acid derivative, such as acipimox, niceritrol or acifran.

### EXAMPLE 7

Pharmaceutical Compositions

Tablets having the composition set forth in Table X-2 are prepared using wet granulation or direct compression techniques:

TABLE X-2

| INGREDIENT | WEIGHT (mg) |
|---|---|
| Eplerenone | 25 |
| Niacin | 250 |
| Lactose | 54 |
| Microcrystalline Cellulose | 15 |
| Hydroxypropyl Methyl Cellulose | 3 |
| Croscarmellose Sodium | 2 |
| Magnesium Stearate | 1 |

### EXAMPLE 8

Pharmaceutical Compositions

Tablets having the composition set forth in Table X-3 are prepared using wet granulation or direct compression techniques:

TABLE X-3

| INGREDIENT | WEIGHT FRACTION (mg) |
|---|---|
| Eplerenone | 50 |
| Acipimox | 200 |
| Lactose | 69.5 |
| Microcrystalline Cellulose | 15 |
| Colloidal Silicon Dioxide | 0.5 |
| Talc | 2.5 |
| Croscarmellose Sodium | 2 |
| Magnesium Stearate | 0.5 |

### EXAMPLE 9

Pharmaceutical Compositions

Tablets having the composition set forth in Table X-4 can be prepared using wet granulation or direct compression techniques:

TABLE X-4

| INGREDIENT | WEIGHT (mg) |
|---|---|
| Eplerenone | 100 |
| Niacin | 250 |
| Lactose | 54 |
| Microcrystalline Cellulose | 15 |
| Hydroxypropyl Methyl Cellulose | 3 |
| Croscarmellose Sodium | 2 |
| Magnesium Stearate | 1 |

The examples herein can be performed by substituting the generically or specifically described reactants and/or operating conditions of this invention for those used in the preceding examples.

In view of the above, it will be seen that the several objects of the invention are achieved. As various changes could be made in the above methods, combinations and compositions of the present invention without departing from the scope of the invention, it is intended that all matter contained in the above description be interpreted as illustrative and not in a limiting sense. All documents mentioned in this application are expressly incorporated by reference as if fully set forth at length.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A combination comprising an aldosterone receptor antagonist and a compound selected from the group consisting of nicotinic acid and nicotinic acid derivatives.

2. The combination of claim 1 wherein the aldosterone recetor antagonist is eplerenone.

3. The combination of claim 1 wherein the aldosterone recetor antagonist is spironolactone.

4. The combination of claim 1 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

5. The combination of claim 2 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

6. The combination of claim 3 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

7. A pharmaceutical composition comprising a first amount of an aldosterone receptor antagonist, a second amount of a compound selected from the group consisting of nicotinic acid and nicotinic acid derivatives, and a pharmaceutically acceptable carrier.

8. The composition of claim 7 wherein the first amount of the aldosterone receptor antagonist and the second amount of a compound selected from the group consisting of nicotinic acid and nicotinic acid derivatives together comprise a therapeutically-effective amount of the aldosterone receptor antagonist and a compound selected from the group consisting of nicotinic acid and nicotinic acid derivatives for the treatment or prophylaxis of a pathogenic condition.

9. The composition of claim 7 wherein said aldosterone receptor antagonist is an epoxy-steroidal-type compound characterized in having a 9α-,11α-substituted epoxy moiety.

10. The composition of claim 9 wherein said epoxy-steroidal-type compound is eplerenone.

11. The composition of claim 7 wherein said aldosterone antagonist is a spirolactone-type compound.

12. The composition of claim 11 wherein said spirolactone-type compound is spironolactone.

13. The composition of claim 7 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

14. The composition of claim 7 wherein said nicotinic acid derivative is selected from the group consisting of niacin, niceritrol, acipimox, and acifran, and the pharmaceutically acceptable salts, esters, conjugate acids, and prodrugs thereof.

15. The composition of claim 7 wherein said nicotinic acid derivative is niacin.

16. The composition of claim 7 wherein said nicotinic acid derivative is niceritrol.

17. The composition of claim 7 wherein said nicotinic acid derivative is acipimox.

18. The composition of claim 7 wherein said nicotinic acid derivative is acifran.

19. The composition of claim 7 wherein said nicotinic acid derivative is cyclohexylphenyl nicotinate.

20. The composition of claim 7 wherein said nicotinic acid derivative is cyclohexylphenyl-oxide nicotinate.

21. The composition of claim 10 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

22. The composition of claim 10 wherein said nicotinic acid derivative is selected from the group consisting of niacin, niceritrol, acipimox, and acifran, and the pharmaceutically acceptable salts, esters, conjugate acids, and prodrugs thereof.

23. The composition of claim 10 wherein said nicotinic acid derivative is niacin.

24. The composition of claim 10 wherein said nicotinic acid derivative is niceritrol.

25. The composition of claim 10 wherein said nicotinic acid derivative is acipimox.

26. The composition of claim 10 wherein said nicotinic acid derivative is acifran.

27. The composition of claim 10 wherein said nicotinic acid derivative is cyclohexylphenyl nicotinate.

28. The composition of claim 10 wherein said nicotinic acid derivative is cyclohexylphenyl-oxide nicotinate.

29. The composition of claim 10 wherein said first amount of eplerenone is between about 0.1 mg to about 400 mg.

30. The composition of claim 10 wherein said first amount of eplerenone is between about 1 mg to about 200 mg.

31. The composition of claim 10 wherein said first amount of eplerenone is between about 1 mg to about 100 mg.

32. The composition of claim 10 wherein said first amount of eplerenone is between about 10 mg to about 100 mg.

33. The composition of claim 10 wherein said first amount of eplerenone is between about 25 mg to about 100 mg.

34. The composition of claim 10 wherein said first amount of eplerenone is selected from the group consisting of about 5 mg, about 10 mg, about 12.5 mg, about 25 mg, about 50 mg, about 75mg, and about 100 mg.

35. The composition of claim 10 wherein said first amount of eplerenone is selected from the group consisting of about 25 mg, about 50 mg and about 100 mg.

36. The composition of claim 12 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

37. The composition of claim 12 wherein said nicotinic acid derivative is selected from the group consisting of niacin, niceritrol, acipimox, and acifran, and the pharmaceutically acceptable salts, esters, conjugate acids, and prodrugs thereof.

38. The composition of claim 12 wherein said nicotinic acid derivative is niacin.

39. The composition of claim 12 wherein said nicotinic acid derivative is niceritrol.

40. The composition of claim 12 wherein said nicotinic acid derivative is acipimox.

41. The composition of claim 12 wherein said nicotinic acid derivative is acifran.

42. The composition of claim 12 wherein said nicotinic acid derivative is cyclohexylphenyl nicotinate.

43. The composition of claim 12 wherein said nicotinic acid derivative is cyclohexylphenyl-oxide nicotinate.

44. A method for treating or preventing a pathogenic condition, said method comprising administering to a subject susceptible to or afflicted with such condition a therapeutically-effective amount of an aldosterone receptor antagonist and a compound selected from the group consisting of nicotinic acid and nicotinic acid derivatives.

45. The method of claim 44 wherein the aldosterone receptor antagonist and the nicotinic acid derivative are administered in a sequential manner.

46. The method of claim 44 wherein the aldosterone receptor antagonist and the nicotinic acid derivative are administered in a substantially simultaneous manner.

47. The method of claim 44, wherein said pathogenic condition is selected from the group consisting of cardiovascular-related conditions, inflammation-related conditions, neurological-related conditions, musculo-skeletal-related conditions, metabolism-related conditions, endocrine-related conditions, dermatologic-related conditions and cancer-related conditions.

48. The method of claim 44, wherein said pathogenic condition is selected from the group consisting of cardiovascular-related conditions.

49. The method of claim 48, wherein said cardiovascular condition is selected from the group consisting of atherosclerosis, hypertension, heart failure, vascular disease, renal dysfunction, stroke, myocardial infarction, endothelial dysfunction, ventricular hypertrophy, renal dysfunction, target-organ damage, thrombosis, cardiac arrhythmia, plaque rupture and aneurysm.

50. The method of claim 44, wherein said pathogenic condition is selected from the group consisting of inflammation-related conditions.

51. The method of claim 50, wherein said inflammatory condition is selected from the group consisting of arthritis, tissue rejection, septic shock, anaphylaxis and tobacco-induced effects.

52. The method of claim 44, wherein said pathogenic condition is selected from the group consisting of neurological-related conditions.

53. The method of claim 52, wherein said neurology-related condition is selected from the group consisting of Alzheimers Disease, dementia, depression, memory loss, drug addiction, drug withdrawal and brain damage.

54. The method of claim 44, wherein said pathogenic condition is selected from the group consisting of musculo-skeletal-related conditions.

55. The method of claim 54, wherein said musculo-skeletal-related condition is selected from the group consisting of osteoporosis and muscle weakness.

56. The method of claim 44, wherein said pathogenic condition is selected from the group consisting of metabolism-related conditions.

57. The method of claim 56, wherein said metabolism-related condition is selected from the group consisting of diabetes, obesity, Syndrome X and cachexia.

58. The method of claim 44, wherein said pathogenic condition is selected from the group consisting of endocrine-related conditions.

59. The method of claim 44, wherein said pathogenic condition is selected from the group consisting of dermatologic-related conditions.

60. The method of claim 44, wherein said pathogenic condition is selected from the group consisting of cancer-related conditions.

61. The method of claim 44, wherein said pathogenic condition is a proliferative disease-related condition.

62. The method of claim 61, wherein said proliferative disease-related condition is cancer.

63. The method of claim 44 wherein said aldosterone receptor antagonist is an epoxy-steroidal-type compound characterized in having a 9α-,11α-substituted epoxy moiety.

64. The method of claim 63 wherein said epoxy-steroidal-type compound is eplerenone.

65. The method of claim 44 wherein said aldosterone antagonist is a spirolactone-type compound.

66. The method of claim 65 wherein said spirolactone-type compound is spironolactone.

67. The method of claim 44 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

68. The method of claim 44 wherein said nicotinic acid derivative is selected from the group consisting of niacin, niceritrol, acipimox, and acifran, and the pharmaceutically acceptable salts, esters, conjugate acids, and prodrugs thereof.

69. The method of claim 44 wherein said nicotinic acid derivative is niacin.

70. The method of claim 44 wherein said nicotinic acid derivative is niceritrol.

71. The method of claim 44 wherein said nicotinic acid derivative is acipimox.

72. The method of claim 44 wherein said nicotinic acid derivative is acifran.

73. The method of claim 44 wherein said nicotinic acid derivative is cyclohexylphenyl nicotinate.

74. The method of claim 44 wherein said nicotinic acid derivative is cyclohexylphenyl-oxide nicotinate.

75. The method of claim 64 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

76. The method of claim 64 wherein said nicotinic acid derivative is selected from the group consisting of niacin, niceritrol, acipimox, and acifran, and the pharmaceutically acceptable salts, esters, conjugate acids, and prodrugs thereof.

77. The method of claim 64 wherein said nicotinic acid derivative is niacin.

78. The method of claim 64 wherein said nicotinic acid derivative is niceritrol.

79. The method of claim 64 wherein said nicotinic acid derivative is acipimox.

80. The method of claim 64 wherein said nicotinic acid derivative is acifran.

81. The method of claim 64 wherein said nicotinic acid derivative is cyclohexylphenyl nicotinate.

82. The method of claim 64 wherein said nicotinic acid derivative is cyclohexylphenyl-oxide nicotinate.

83. The method of claim 64 wherein said eplerenone is administered in a daily dose range between about 0.1 mg to about 400 mg.

84. The method of claim 64 wherein said eplerenone is administered in a daily dose range between about 1 mg to about 200 mg.

85. The method of claim 64 wherein said eplerenone is administered in a daily dose range between about 1 mg to about 100 mg.

86. The method of claim 64 wherein said eplerenone is administered in a daily dose range between about 10 mg to about 100 mg.

87. The method of claim 64 wherein said eplerenone is administered in a daily dose range between about 25 mg to about 100 mg.

88. The method of claim 64 wherein said eplerenone is administered in a daily dose selected from the group consisting of about 5 mg, about 10 mg, about 12.5 mg, about 25 mg, about 50 mg, about 75 mg, and about 100 mg.

89. The method of claim 64 wherein said eplerenone is administered in a daily dose selected from the group consisting of about 25 mg, about 50 mg and about 100 mg.

90. The method of claim 66 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

91. The method of claim 66 wherein said nicotinic acid derivative is selected from the group consisting of niacin, niceritrol, acipimox, and acifran, and the pharmaceutically acceptable salts, esters, conjugate acids, and prodrugs thereof.

92. The method of claim 66 wherein said nicotinic acid derivative is niacin.

93. The method of claim 66 wherein said nicotinic acid derivative is niceritrol.

94. The method of claim 66 wherein said nicotinic acid derivative is acipimox.

95. The method of claim 66 wherein said nicotinic acid derivative is acifran.

96. The method of claim 66 wherein said nicotinic acid derivative is cyclohexylphenyl nicotinate.

97. The method of claim 66 wherein said nicotinic acid derivative is cyclohexylphenyl-oxide nicotinate.

98. A kit for treating or preventing a pathogenic condition comprising an aldosterone receptor antagonist and a compound selected from the group consisting of nicotinic acid and nicotinic acid derivatives.

99. The kit of claim 98 wherein said aldosterone receptor antagonist is an epoxy-steroidal-type compound characterized in having a 9α-,1α-substituted epoxy moiety.

100. The kit of claim 99 wherein said epoxy-steroidal-type compound is eplerenone.

101. The kit of claim 98 wherein said aldosterone antagonist is a spirolactone-type compound.

102. The kit of claim 101 wherein said spirolactone-type compound is spironolactone.

103. The kit of claim 98 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

104. The kit of claim 98 wherein said nicotinic acid derivative is selected from the group consisting of niacin, niceritrol, acipimox, and acifran, and the pharmaceutically acceptable salts, esters, conjugate acids, and prodrugs thereof.

105. The kit of claim 98 wherein said nicotinic acid derivative is niacin.

106. The kit of claim 98 wherein said nicotinic acid derivative is niceritrol.

107. The kit of claim 98 wherein said nicotinic acid derivative is acifran.

108. The kit of claim 98 wherein said nicotinic acid derivative is ciprofibrate.

109. The kit of claim 98 wherein said nicotinic acid derivative is cyclohexylphenyl nicotinate.

110. The kit of claim 98 wherein said nicotinic acid derivative is cyclohexylphenyl-oxide nicotinate.

111. The kit of claim 100 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

112. The kit of claim 100 wherein said nicotinic acid derivative is selected from the group consisting of niacin, niceritrol, acipimox, and acifran, and the pharmaceutically acceptable salts, esters, conjugate acids, and prodrugs thereof.

113. The kit of claim 100 wherein said nicotinic acid derivative is niacin.

114. The kit of claim 100 wherein said nicotinic acid derivative is niceritrol.

115. The kit of claim 100 wherein said nicotinic acid derivative is acipimox.

116. The kit of claim 100 wherein said nicotinic acid derivative is acifran.

117. The kit of claim 100 wherein said nicotinic acid derivative is cyclohexylphenyl nicotinate.

118. The kit of claim 100 wherein said nicotinic acid derivative is cyclohexylphenyl-oxide nicotinate.

119. The kit of claim 102 wherein said nicotinic acid derivative is selected from the group consisting of niacin, niceritrol, acipimox, acifran, cyclohexylphenyl nicotinate, and cyclohexylphenyl-oxide nicotinate, and the pharmaceutically acceptable salts, esters, conjugate acids, and prodrugs thereof.

120. The kit of claim 102 wherein said nicotinic acid derivative is selected from the group consisting of niacin, niceritrol, acipimox, and acifran, and the pharmaceutically acceptable salts, esters, conjugate acids, and prodrugs thereof.

121. The kit of claim 102 wherein said nicotinic acid derivative is niacin.

122. The kit of claim 102 wherein said nicotinic acid derivative is niceritrol.

123. The kit of claim 102 wherein said nicotinic acid derivative is acipimox.

124. The kit of claim 102 wherein said nicotinic acid derivative is acifran.

125. The kit of claim 102 wherein said nicotinic acid derivative is cyclohexylphenyl nicotinate.

126. The kit of claim 102 wherein said nicotinic acid derivative is cyclohexylphenyl-oxide nicotinate.

127. The kit of claim 98 further comprising written instructions for the use of said kit by a subject.

128. The kit of claim 127 wherein the written instructions state how the subject can use said kit to obtain a therapeutic effect without inducing unwanted side-effects.

129. The kit of claim 127 wherein the written instructions comprise all or a part of the product label approved by a drug regulatory agency for said kit.
